(19) United States
(12) Patent Application Publication
(10) Pub. No.: US 2010/0057046 A1
(43) Pub. Date: Mar. 4, 2010

(54) SYSTEMS FOR CHARACTERIZING PHYSIOLOGIC PARAMETERS AND METHODS FOR USE THEREWITH

(75) Inventors: Margaret R. Stevens (nee Webber), Los Altos, CA (US); Richard Blakley, Livermore, CA (US); Hank Zoeller, Sunnyvale, CA (US)

Correspondence Address: KNOBBE MARTENS OLSON & BEAR LLP, 2040 MAIN STREET, FOURTEENTH FLOOR, IRVINE, CA, 92614, US

(73) Assignee: KEIMAR, INC, Pleasanton, CA (US)

(21) Appl. No.: 12/552,081

(22) Filed: Sep. 1, 2009

Related U.S. Application Data
(60) Provisional application No. 61/094,033, filed on Sep. 3, 2008.

(51) Int. Cl. A61M 16/00; A61M 37/00

(52) U.S. Cl. 604/507; 604/66; 128/204.23

(57) ABSTRACT

Disclosed herein are systems and methods for continuously measuring a physiologic parameter of a patient, and can also include adjusting therapy based upon the physiologic parameter. The system can include a first probe having an elongate body, the probe configured to be inserted into a first location within a patient. At least one sensor can be operably connected to the first probe and configured to continuously provide real-time feedback information on one or more physiologic parameters at the first location within the patient, such as pH, pCO₂, pO₂, pressure, or temperature. A controller can be connected to the probe and configured to receive the real-time feedback information and to adjust a therapeutic setting on a therapeutic device based at least in part on the feedback information.

### CROSS-REFERENCE TO RELATED APPLICATIONS

This application claims priority benefit under 35 U.S.C. §119(e) to U.S. Provisional App. No. 61/094,033 filed on Sep. 3, 2008, which is hereby incorporated by reference in its entirety.

### BACKGROUND

1. Technical Field

This application relates to probes and sensors for measuring physiological parameters in a body such as a mammalian body and, in particular, to probes for ascertaining characteristics of blood in a body such as a mammalian body.

2. Description of the Related Art

Determination of cardiac output, arterial blood gases, blood pressure and other hemodynamic or cardiovascular parameters is critically important in the treatment and care of patients, particularly those undergoing surgery or other complicated medical procedures and those under intensive care. Such parameters provide important patient status information to caregivers that can inform treatment parameters and decisions.

Typically, cardiac output measurements have been made using pulmonary artery thermodilution catheters, which can have inaccuracies of 20% or greater. It has been found that the use of such thermodilution catheters increases hospital costs while exposing the patient to potential infectious, arrhythmogenic, mechanical, and therapeutic misadventure. Blood gas measurements have also heretofore been made. Commonly used blood gas measurement techniques require a blood sample to be removed from the patient and transported to a lab analyzer for analysis. The caregiver must then wait for the results to be reported by the lab, a delay of 20 minutes being typical and longer waits not unusual.

“Point-of-care” blood testing systems allow blood sample analysis at a patient's bedside or in the area where the patient is located. Such systems include portable and handheld units and modular units that fit into a bedside monitor and can determine parameters such as metabolite and blood gas concentrations. While most point-of-care systems require the removal of blood from the patient for bedside analysis, a few do not. In some systems, intermittent blood gas and metabolite measurements are made by drawing a sufficiently large blood sample into an arterial line to ensure an undiluted sample at a sensor located in the line. After analysis, the blood is returned to the patient, the line is flushed, and results appear on the bedside monitor. In other systems, such as those that measure the concentration of single or multiple metabolites in a patient's blood, blood is drawn into a syringe and placed into a vial or ampule, microfuged to separate plasma from platelets, and pipetted into a sample vial that is placed into a bench-top or floor-model analyzer for measurement. Such analyzers require many operating steps and are expensive, bulky, and not readily accessible, practical, or affordable in many situations and settings.

A non-invasive technology, pulse oximetry, is available for estimating the percentage of hemoglobin in arterial blood that is saturated with oxygen. Although pulse oximeters are capable of estimating arterial blood oxygen content, they are not capable of measuring carbon dioxide, pH, the partial pressure of oxygen, or venous oxygen content. Furthermore, pulse oximetry is commonly performed at the fingertip and can be skewed by peripheral vasoconstriction or even nail polish. Although pulse oximetry can also be used to measure blood metabolite concentrations, such measurements are not as precise and reliable as electrochemical measurements for similar reasons.

Blood pressure can be measured non-invasively using a blood pressure manometer connected to an inflatable cuff. This is the most common method outside of the intensive care environment. In critical care settings, at least 60% of patients have arterial lines. An arterial line can include a plastic cannula inserted into a peripheral artery (commonly the radial or the femoral). The cannula is kept open and patent because it is connected to a pressurized bag of heparinized fluid such as normal saline. An external gauge also connects to the arterial cannula to reflect the column of fluid pressure in the artery. This system consists of an arterial line connected to a pressure transducer by saline-filled, non-compressible tubing. This converts the pressure waveform into an electrical signal which is displayed on the bedside monitor. The pressurized saline for flushing is provided by a pressure bag. Several potential sources of error exist in this system. First, any one of the many components in the system can fail. Second, the transducer position is critical because the pressure displayed is pressure relative to position of transducer. Thus, in order to accurately reflect blood pressure, the transducer should be at the level of the heart. Over-reading will occur if transducer too low and under-reading if transducer too high. Third, the transducer must be zeroed to the atmospheric pressure at the time of measurement, otherwise the blood pressure will be incorrectly measured. Fourth, it is critical to have appropriate damping in the system. Inadequate damping will result in excessive resonance in the system, which causes an overestimate of systolic pressure and an underestimate of diastolic pressure. An under-damped trace is often characterized by a high initial spike in the waveform. The opposite occurs with over-damping. In both cases, the mean arterial pressure is the most accurate.

Closed-loop systems provide a platform for directing treatment based on feedback from sensors such as those specifically described in the present disclosure. Treatment is typically provided by a device that is most effective when continually adjusted in response to changing patient conditions.

Unfortunately, none of the available systems or methods for blood gas analysis provides for a reliable, closed-loop system having accurate, direct and continuous in vivo measurements of arterial and venous oxygen partial pressures, carbon-dioxide partial pressure, pH, cardiac output, and blood pressure while presenting minimal risk to the patient.

### SUMMARY OF THE INVENTION

Probes and sensors for characterizing fluids, and systems and methods for use therewith, are described. Immersible probes having one or more separately contained sensors are employed in closed-loop systems to provide feedback useful in setting control parameters in medical treatment regimes. The disclosed closed-loop systems and methods can replace or supplement conventional treatment monitoring and adjustment protocols. The disclosed probes and sensors provide an ideal, versatile, robust, and reliable platform for use alone or in conjunction with the systems described.

A probe is provided for use in a patient to ascertain characteristics of the patient's blood and other fluids such as recuperated air. The probe comprises a cannula adapted to be inserted into the patient's blood vessel. The probe carries various sensors, which can be utilized in various other environments. A gas, metabolite and/or pressure sensor assembly, of one or more sensors, is carried within the distal extremity of the cannula for determining gas characteristics, metabolite concentrations, and/or pressure of the blood. Signal processing or analysis is employed to determine parameters that depend on characteristics ascertained by the sensor or sensors. Such determinations can be used in feedback systems to supplement or replace human control and/or monitoring either at or remote from the point of care. The disclosed sensor assembly is especially useful in endovascular and tissue measurements.

Closed-loop systems that utilize sensors and probes for blood and other fluids such as expired air are described. The probes and sensors provide feedback that is used in the control of a device providing treatment. The device can be a respirator, kidney dialysis machine, implantable or external drug pump, or any other such device. Such devices can be electronically or mechanically controlled by computer or human inputs or a combination of both. Treatment decisions that are informed in the present closed-loop systems and methods include whether to provide ventilation to a patient who cannot self-ventilate, and, if so, at what frequency and intensity and for what duration, whether to provide insulin to a diabetic patient and, if so, when and how much, and whether a patient with a pacemaker should be paced differently based on the adequacy of perfusion.

Also disclosed herein is a medical system for continuously measuring a physiologic parameter of a patient and adjusting therapy based upon feedback information on the physiologic parameter. In some embodiments, the system includes a first probe having an elongate body. The probe is configured to be inserted into a first location. Also included is a first sensor operably connected to the first probe and configured to continuously provide feedback information on at least one physiologic parameter at the first location. The physiologic parameter can include, for example, pH, pCO₂, pO₂, pressure, and temperature. The system can also include a controller operably connected to the probe. The controller can be configured to receive the feedback information and to adjust a therapeutic setting on a therapeutic device based at least in part on the feedback information. The first probe can include, for example, a catheter, a wire, or a pacer lead. The first sensor can be part of an array of sensors operably connected to the first probe. The feedback information can be real-time or delayed feedback information. The controller can be configured to communicate with the therapeutic device via a physical connection, or wirelessly communicate with the therapeutic device in other embodiments. The first or a second location can be, for example, an anatomical location of the patient, such as within the arterial circulation or venous circulation, within a ventricle of the brain, or within the left or right side of a patient's heart. The system can also be located, for example, within an endotracheal tube having a distal end residing within the patient's respiratory tract, or within a conduit of a cardiopulmonary bypass loop. The therapeutic device can be an infusion device for medications, solutions, or both. The therapeutic device can also be a ventilator. The therapeutic setting could be, for example, a setting affecting minute ventilation, such as ventilatory rate and/or tidal volume. The therapeutic setting can be FiO₂, positive end-expiratory pressure, or pressure support in other embodiments. The ventilator could be either invasive or non-invasive. In some embodiments, the system also includes a second probe having an elongate body, the second probe configured to be inserted into a second location within a patient; and at least a second sensor operably connected to the second probe and configured to continuously provide real-time feedback information on at least one physiologic parameter at the second location within the patient. The physiologic parameters can be one or more of, for example, pH, pCO₂, pO₂, pressure, or temperature. In some embodiments, the system also includes a module configured to determine the cardiac output of a patient based at least in part from the feedback information from the first sensor and the second sensor. The module could be part of the controller, or a discrete unit in other embodiments.

In another embodiment, also disclosed herein is a medical system for continuously measuring a physiologic parameter of a patient and adjusting therapy based upon feedback information on the physiologic parameter. The system includes a first probe having an elongate body, the probe configured to be inserted into a first location within the arterial circulation of a patient. The system also includes a sensor array operably connected to the first probe and configured to continuously provide feedback information on at least one physiologic parameter at the first location within the patient, the at least one physiologic parameter including, for example, pH, pCO₂, pO₂, pressure, or temperature. The system can also include a controller configured to operably communicate with the probe, and a module configured to calculate a patient's cardiac output based in at least in part upon feedback information from the sensor array. The controller can be configured to receive the feedback information and to adjust a therapeutic setting on a ventilator based at least in part on the feedback information.

Also disclosed herein is a method of continuously monitoring at least one physiologic parameter of a patient. The method can include the steps of providing a first probe having an elongate body, the probe configured to be inserted into a first location, and the probe operably connected to a first sensor. The probe is then delivered to the first location, which can be, in some embodiments, the arterial or venous circulation of the patient, the left heart, right heart, inside the cranium, the abdominal cavity, or a cavity within an extremity. The first location could also be a non-anatomical location such as within an endotracheal tube or within a conduit of a cardiopulmonary bypass system. The at least one physiologic parameter of the patient is continuously measured at the first location using the first sensor. The at least one physiologic parameter can be, for example, one or more of pH, pCO₂, pO₂, pressure, and temperature. Real-time or delayed feedback information regarding the physiologic parameter can then be transmitted to an output device. The probe can be, in some embodiments, a guidewire, pacer wire, or catheter. The method can also include interpreting the physiologic parameter feedback information; and adjusting a therapeutic setting on a therapeutic device at least partially based on the physiologic parameter feedback information from the first location. Interpreting the physiologic parameter feedback information can be performed by a controller, or a health care provider in some embodiments. Adjusting a therapeutic setting on a therapeutic device can include, in some embodiments, sending instructions from the controller to the therapeutic device.

The method can also include providing a second probe having an elongate body, the second probe configured to be inserted into a second location to measure at least one physiologic parameter of the patient, the second probe operably connected to a second sensor configured to continuously provide feedback information on the at least one physiologic parameter at the second location within the patient. The method can also include delivering the second probe to a second anatomical location of the patient, and continuously measuring the at least one physiologic parameter of the patient at the second location using the second sensor. In some embodiments, the method includes transmitting physiologic parameter feedback information from the second location to a second output device, which can be the same device or a different device than the first output device. The second anatomical location could be, for example, one of the locations discussed for the first anatomical locations above. In some embodiments, the method includes determining the patient's cardiac output using the physiologic parameter feedback from the first location and from the second location.

### BRIEF DESCRIPTION OF THE DRAWINGS

For a better understanding of the nature and details of the present inventions, reference can be made to the following drawings, which, in some instances, are schematic in detail.

FIG. 1 is an isometric view of an example embodiment of a probe for ascertaining blood characteristics coupled to a display module.

FIG. 2 is a cutaway and partially sectioned view of the connector portion of one embodiment of a probe.

FIGS. 2A, 2B, and 2C are a section or plan view and two plan views, respectively, of an alternative and preferred version of the connector portion of one embodiment of a probe.

FIG. 3 is an enlarged, cross-sectional view of the pH sensor section of one embodiment of a probe.

FIG. 4 is an enlarged, cross-sectional view of the carbon dioxide sensor section of one embodiment of a probe.

FIG. 5 is an enlarged, cross-sectional view of the oxygen sensor section of one embodiment of a probe.

FIG. 5A is an enlarged, cross-sectional view of an alternative and preferred version of the oxygen sensor section of one embodiment of a probe.

FIG. 6A is an enlarged, cross-sectional view of one embodiment of the blood pressure sensor section of a probe.

FIG. 6B is a cross-sectional view of the blood pressure sensor section, orthogonal to FIG. 6A.

FIG. 6C is a cross-sectional view of the blood pressure sensor section, orthogonal to FIGS. 6A and 6B.

FIG. 7A shows a method of using one embodiment, and various components of an example of a system including a feedback loop.

FIG. 7B illustrates a catheter-mounted or catheter-embedded embodiment.

FIG. 7C illustrates a portion of a catheter-mounted or catheter-embedded embodiment.

FIG. 8 is a plan or sectional view of the top of an embodiment of the probe.

FIG. 9 is a plan view of the bottom of the first layer of an embodiment of the probe.

FIG. 10 is a plan view of the second layer of an embodiment of the probe.

FIG. 11 is a plan view of the top of the third layer of an embodiment of the probe.

FIG. 12 is a bottom plan view of an embodiment of the probe.

FIG. 13 is a bottom plan view of an embodiment of the probe.

FIG. 14 is an isometric view of another embodiment of a probe for ascertaining blood characteristics.

FIG. 15 is a plan view, partially cut away, of an example embodiment of a kit.

FIGS. 16 and 16A show an example embodiment of a sensor disposed within a patient and a portion thereof, respectively.

FIG. 17 shows a schematic of a closed-loop system.

### DETAILED DESCRIPTION

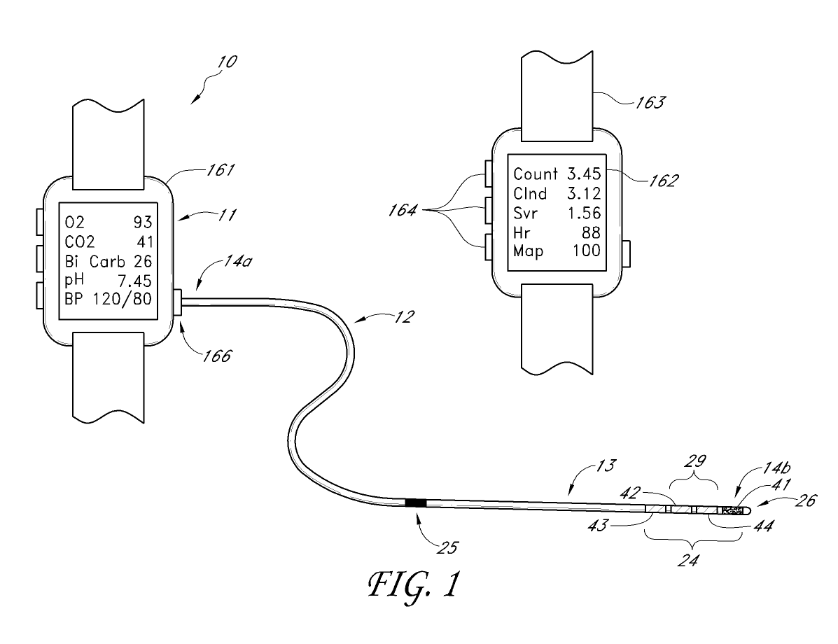

An apparatus 10 according one embodiment of the present invention for making intravascular measurement of physiological parameters or characteristics generally includes, as shown in FIG. 1, a display module 11 and one or more probes 12. As described in more detail herein, the display module 11 and probe 12 are particularly adapted for accurate and continuous in vivo measurement and display of intravascular parameters such as, for example, partial pressure of oxygen (pO₂), partial pressure of carbon dioxide (pCO₂), pH, temperature, and blood pressure. In some embodiments, the probes as described herein can be configured to continuously sense one, two, or more physiologic parameters for at least about 5, 10, 15, 20, 30, or 45 minutes, or at least about 1, 2, 3, 4, 5, 6, 8, 10, 12 hours, or at least about 1, 2, 3, 4, 5, 6, days, or at least about 1 week, 2 weeks, 1 month, 3 months, 6 months, 1 year, or more. Continuous sensing as described herein includes sensing without interruption or substantially without interruption. Continuous sensing can also encompass repeated intermittent sensing of the physiologic parameters at specified intervals, such as at least about every 15 minutes, 10 minutes, 5 minutes, 3 minutes, 2 minutes, 1 minute, 30 seconds, 15 seconds, 10 seconds, 5 seconds, 2 seconds, 1 second, 0.5 seconds, 0.25 seconds, 0.1 second, 0.05 seconds, 0.01 seconds, or more frequently.

Cardiac output (CO) can be calculated by combining two pO₂ measurements obtained from a pair of probes, one disposed in an artery and the other in a vein. Alternatively, or in addition to the aforementioned sensors, the probe 12 may include sensors for other blood parameters such as potassium, sodium, calcium, bicarbonate, urea nitrogen, creatinine, bilirubin, hemoglobin, glucose, and lactate. Additional features of some embodiments of display modules, probe, and sensors are detailed hereinafter and in U.S. patent application Ser. No. 12/172,181, filed on Jul. 11, 2008; U.S. patent application Ser. No. 12/027,933, filed on Feb. 7, 2008; U.S. patent application Ser. No. 12/027,915, filed on Feb. 7, 2008; U.S. patent application Ser. No. 12/027,902, filed on Feb. 7, 2008; U.S. patent application Ser. No. 12/027,898, filed on Feb. 7, 2008; U.S. patent application Ser. No. 12/027,905, filed on Feb. 7, 2008; U.S. patent application Ser. No. 10/658,926, filed on Sep. 9, 2003; U.S. Provisional App. No. 61/152,183, filed on Feb. 12, 2009; and U.S. Pat. No. 6,616,614, each of which is hereby each incorporated by reference in its entirety. It will be appreciated that the systems and clinical applications described herein are not limited to any particular sensor or probe described herein. Rather, any suitable sensor or probe, for example, but not limited, to those described in the incorporated applications, may also be used.

FIG. 1 illustrates a probe 12 comprising a flexible elongate probe body, cannula, or sleeve 13. The cannula 13 may be formed of a suitable insulating material such as a plastic, which provides strength and flexibility to the cannula 13 and thus may serve as a structural element of the probe 12. An example material for the cannula 13 includes a polymer such as polymethylpentene. Among commonly-used polymers suitable for extrusion as thin-walled tubing, polymethylpentene has among the highest oxygen and carbon dioxide permeability coefficients available, along with great stiffness. The cannula 13 has a proximal extremity or end portion 14a and a distal extremity or end portion 14b, and has a substantially uniform diameter or cross-sectional area over its entire length. In some embodiments, the cannula 13 has a wall thickness ranging from, for example, about 0.001 inches to about 0.003 inches and, in some embodiments, is approximately 0.0015 inches. The cannula 13 is long enough so that when the distal extremity 14b is in a vessel of a patient, the proximal extremity 14a is accessible outside of the body of the patient. The probe 12 includes a sensor section 24, a marker band 25, and a blunt tip 26 at the distal end portion or extremity 14b.

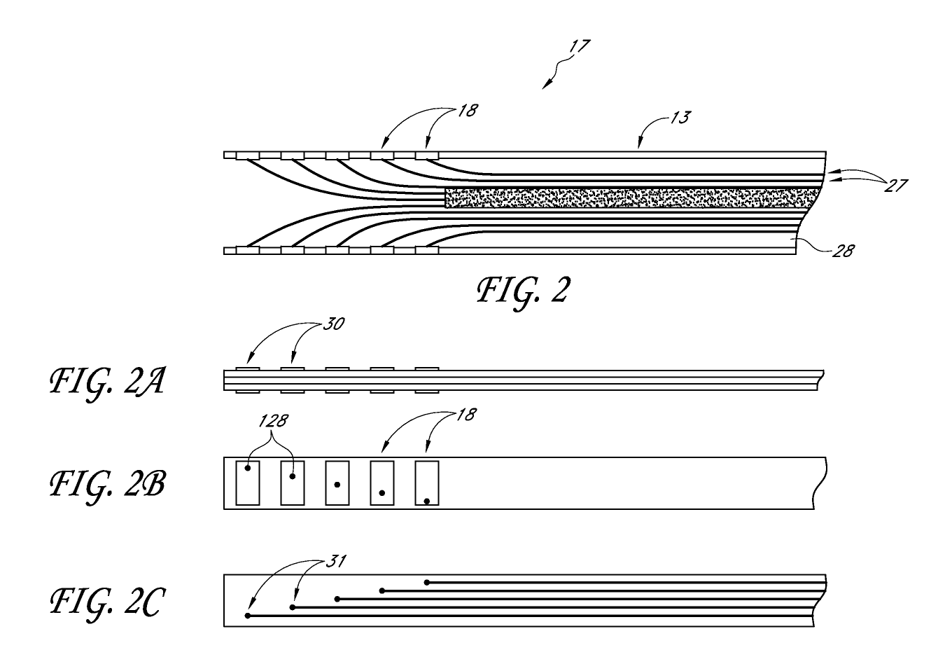

The probe 12 is configured to removably connect to and communicate with a display module 11, for example by way of a suitable probe connector 17, shown in FIG. 2, located at the proximal end 14a and having a plurality of electrical contacts 18 that are annularly or cylindrically disposed on the probe 12. Other suitable bands or pads are also possible. For example, the electrical contacts 18 may also be distributed on one or both sides of a flat connector, such as a flexible printed circuit board. Certain such electrical contacts 18 can provide for a low-profile or even zero-profile electrical connector 17. The electrical contacts 18 may comprise a conductor, such as gold. A plurality of electrical conductors or conductor means 27 pass through the length of the cannula 13, through a bore or lumen 28, provided in the tubular cannula, and attach to the plurality of contacts 18 of the connector 17 for providing electrical outputs to the proximal extremity 14a of cannula 13. The conductors 27 can be formed from any suitable conductive material such as copper, platinum, or silver, which is covered by an insulating material and are of uniform diameter or thickness along their entire length of the conductor extending between the exposed ends. The contacts 18 are soldered, welded, or otherwise electrically coupled to the electrical conductors 27, which are electrically coupled to the one or more sensors in the sensor section 24 of the probe 12 so as to carry electrical signals from such multiple sensors, and thus permit electrical access to the probe 12 from outside the patient's body. Alternatively, the electrical conductors 27 are formed of specific conductive materials such as platinum or silver, the distal ends of which are formed into the various sensor elements.

One embodiment of probe connector 17 is shown in FIGS. 2A, 2B, and 2C. FIG. 2A shows a cross-sectional or plan view of three layers; each layer is formed of a suitable insulating sheet, such as that used for flexible printed circuits. The top and bottom layers, shown in FIGS. 2B and 2C, respectively, are each plated with suitable conductive material in the form of traces 31 and pads 30. The traces 31 are connected at their distal ends to electrical conductors 27 by soldering or other conductive means. The traces 31 are connected at their proximal ends to pads 30 on the reverse side of the layer by means of plated vias 128 through the layer, in a conventional means used for flexible printed circuits. The three layers are bonded together as shown in FIG. 2A, in a conventional manner used for flexible printed circuits.

Referring back to FIG. 1, a gas permeable window 29 preferably covers at least the oxygen and carbon dioxide portions of the sensor section 24 of the probe 12. In this regard, all or a portion of the cannula 13 can also serve as a gas-permeable membrane or window 29. When serving as the gas permeable membrane, the polymer material of the cannula 13 permits the passage of oxygen and carbon dioxide gases while blocking the passage of liquid water and the ions dissolved therein. The cannula 13 defines the outer surface of a major portion of the probe 12, and the substantial majority of the cannula 13 can be filled with a flexible polymer 33, for example ultraviolet-cured adhesive (also referred to as adhesive encapsulant 33), to provide robustness to the probe body 13, to anchor the electrical conductors 27 and sensor electrode assemblies inside the sensor section 24, and to seal the ends of any chambers provided in the probe 12 in the vicinity of such sensor electrode assemblies. In some embodiments, multiple types of adhesive 33 and other fillers may be utilized to improve either the performance or the ease of assembly of the probe 12. For example, cyanoacrylate can be used for small-scale bonding and small gap filling and an ultraviolet-cured adhesive can be used for large gap filling and forming chamber walls.

All of the probe elements are dimensioned to fit substantially within the diameter of the probe body 13 such that the entire probe 12, including the low-profile connector 17, may be passed through the inner bore of a suitable introducer, such as a hypodermic needle (not shown), of a size suitable for accessing a blood vessel in the hand, wrist, or forearm. In some embodiments, the probe body 13 has an outer diameter in the range of about 0.015 inches to about 0.030 inches. In some embodiments, the probe body 13 has an outer diameter of approximately 0.020 inches. Depending on the diameter of the probe body 13, a suitable hypodermic needle for this purpose may be 20-gauge with an inner diameter of at least 0.023 inches, suitable for use with a probe body having a nominal diameter of 0.020 inches. In some embodiments, the probe 12 can have a suitable length such as 25 centimeters, permitting the sensor section 24 to be inserted into a blood vessel in the hand, wrist, or forearm, while the low-profile connector 17 at the proximal end or extremity of probe 12 is connected to the display module 11, which can be strapped to the patient's wrist. Marker band 25 is a guide for the insertion of the probe 12, and is placed, for example, 50 millimeters from the distal end of extremity 14b of the probe 12. When the probe 12 is completely inserted, marker band 25 should be visible just outside the access point of the probe 12 into the skin.

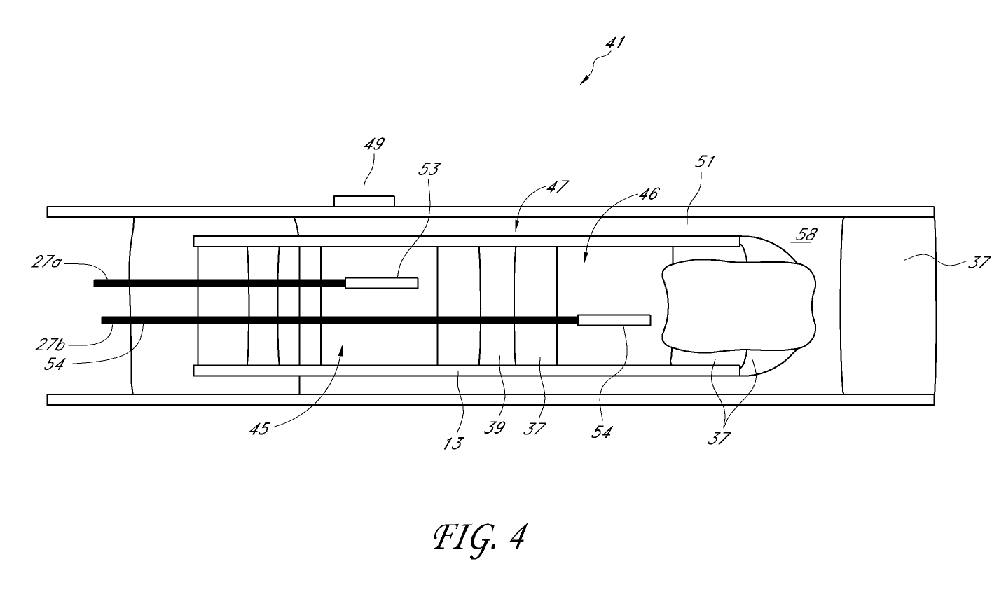

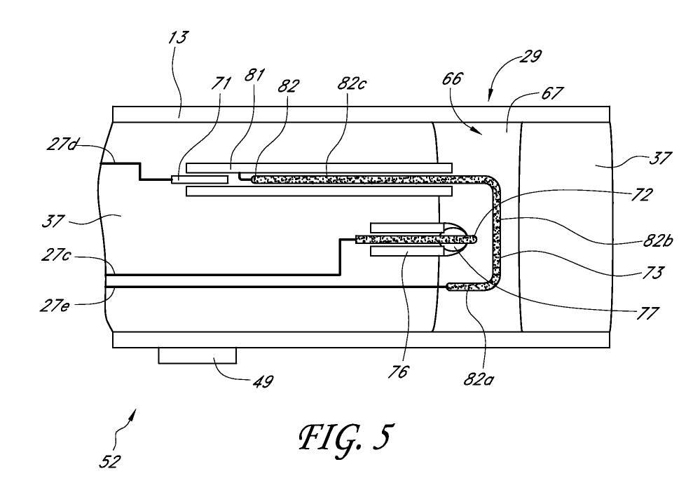

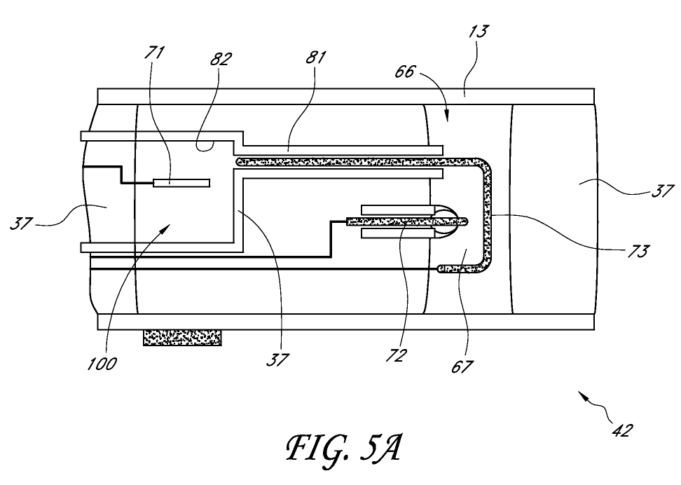

At least one sensor is carried by distal extremity 14b of cannula 13 in the sensor section 24 of probe 12. The sensor section 24 of the probe 12 includes electrodes inside at least one electrolyte-filled chamber. Such multiple sensors can include a carbon dioxide sensor 41, an oxygen sensor 42, a pressure sensor 43, a pH-sensing electrode 44, or any combination thereof or other sensors, for example a temperature sensor. Some or all of the sensors can be utilized for determining gas characteristics of the blood in a vessel. The sensors, separately or combined, are sometimes referred to herein as a sensor assembly. In some embodiments, at least the portion of the cannula 13 that is placed inside the blood vessel, including the sensor section 24, is provided with a surface treatment 49, a portion of which is shown in FIGS. 4 and 5, to inhibit the accumulation of thrombus, protein, or other blood components which might otherwise impair the blood flow in the vessel or impede the diffusion of target analyte into the chambers of the sensor section 24.

The individual sensors of sensor section 24 each occupy a small axial length of the probe 12 in the range, for example, about 5 mm to about 10 mm, and, in some embodiments, approximately 6 mm, so that the sensor section 24 of the probe 12 is relatively short, such as less than 25 mm, to be easily advanced into a tortuous vessel.

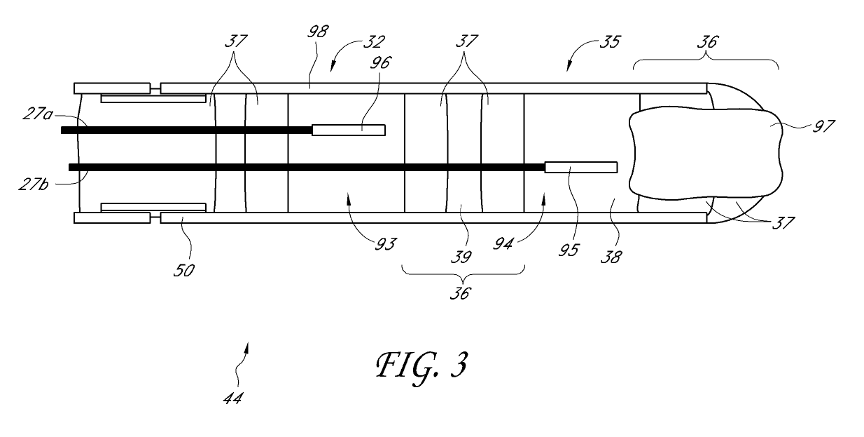

The pH sensor 44, shown in detail in FIG. 3, is carried by distal extremity 14b of the cannula 13 and is contained within the sensor section 24 of probe 12. As shown in FIG. 3, there are two cells: the potential of one cell is selectively dependent on the pH of the blood surrounding probe 12 (the working or pH sensing cell 32) and the reference cell 35 provides a reference potential (the voltage reference cell 94). The pH sensor 44 functions like any classic pH sensor. The pH-sensing electrode 96 is of sufficient area to generate a measurable pH-dependent potential. Measurement of the potential of the pH-sensing electrode 96, with respect to the potential of the voltage reference electrode 95, allows quantification of the pH of the blood that is in contact with the frit 97 and the external surfaces of the walls of the chamber surrounding pH-sensing electrode 96.

The two cells (32, 35) are separated from each other and from the rest of the sensors in probe 12 by insulating walls 36; each insulating wall 36 comprises one or more layers of insulators 37, such as adhesive encapsulant 37, encapsulated air 39, and/or other insulating material.

The most distal cell of pH sensor 44 is the voltage reference cell 35 and comprises a chamber 94, an electrolyte solution or conductive gel 38 filling the chamber 94, defined by an inner wall of the pH-sensitive glass tube 50, a reference electrode 95, which is immersed in this solution or gel 46, and a frit 97. Depending on material and/or performance requirements, the pH-sensitive glass tube 50 could be substituted with another pH-sensitive material such as a material comprising an ion-selective transporter. The electrode 95 can be formed from a silver wire which is coated with silver chloride at its distal end, produced by dipping the silver wire into molten silver chloride or by another electrochemical process. The cylindrical wall 39 of chamber 94 is of any material such as glass or plastic which is relatively impermeable to gases in the blood. Embedded in the adhesive encapsulant 37 which seals the distal end of chamber 94 is a frit 97, composed of an appropriate porous material such as ceramic or glass, such as Vycor 7930. The distal end of the frit 97 is exposed to blood; the proximal end of the frit 97 is exposed to the electrolyte solution or conductive gel which fills chamber 94. The porous frit 97 provides a liquid junction between the blood on the outside of probe 12 and the solution or gel 38 which fills chamber 94 while limiting leeching of the solution or gel 38 from the chamber 94.

The pH-sensing cell 32 of pH sensor 44 is just proximal to the voltage reference cell 35, separated by an insulating wall 36. The pH-sensing cell 32 comprises a chamber 93, a pH buffered solution 99, a pH-sensing electrode 96, and cylindrical walls defined by pH sensitive glass 98. The pH-sensing electrode 96 is formed in the same way as the voltage reference electrode 95 is formed, and is immersed in the pH buffered solution 99 that fills the chamber 93.

The pH-sensing electrode 96 is attached to an electrical conductor 27g, such as an insulated copper or platinum wire, by soldering or welding. The portion of conductor 27g extending from electrode 96 through the chamber 48 and back to the connector 17 is covered with any suitable insulating material. The voltage reference electrode 95 is attached to an electrical conductor 27h, such as an insulated copper or platinum wire, by soldering or welding. The portion of conductor 27h extending from electrode 95 through the chamber 94 and chamber 48 and back to the connector 17 is covered with any suitable insulating material. Alternatively, the conductors 27g and 27h are silver wires, the distal ends of which are formed into electrodes 96 and 95.

A detailed view of one embodiment of the carbon dioxide sensor 41 suitable to be contained within the sensor section 24 of probe 12 is shown in FIG. 4. The carbon dioxide sensor 41 resembles the pH sensor 44 and has a carbon-dioxide-sensing element 42 comprising an electrode 53, which is suspended in a chamber 51. Adhesive encapsulant 37 seals each end of chamber 51 and secures the proximal end of the carbon-dioxide-sensing element 42. The chamber 51 is preferably filled with an electrolyte solution 58 such as a mixture of 0.154 molar NaCl (normal saline) and 0.026M NaHCO₃ (sodium bicarbonate). The cells, electrodes and conductive elements for the carbon-dioxide-sensing element 42 are made with the same methods as the cells, electrodes and conductive elements as are used to construct the pH sensor 44. Conductors 27a and 27b are connected to the sensing electrode 53 and the reference electrode 54, respectively, of the carbon dioxide sensor 41 in the same way that their counterparts are connected to the electrodes of the pH sensor 44.

As with the pH sensor 44, the pH-sensing cell 45 of the carbon-dioxide sensor 41 generates a measurable pH-dependent potential and the voltage reference cell 46 generates a potential that is essentially independent of pH. Carbon dioxide gas permeation through a polymethylpentene membrane (not shown) of the cannula 13 of some embodiments results in a pH change in the electrolyte solution 58 which, in turn, causes a change in potential at the pH-sensing electrode 53. This change in potential is proportional to the carbon dioxide partial pressure in the blood surrounding probe 12. Thus, measurement of the potential at the pH-sensing electrode 53 in addition to the potential of the reference electrode 54 of the voltage reference cell allows quantification of the partial pressure of carbon dioxide in the blood outside the probe 12.

One embodiment of the oxygen sensor 52 is illustrated in FIG. 5, which comprises an oxygen main chamber 66 containing an electrolyte solution 67, a first or reference electrode 71, a second or working electrode 72 and a third or counter electrode 73. The main chamber 66 is defined by the cannula or sleeve 13 and the adhesive encapsulant 37, which seals each end of the chamber 66. The main chamber 66 is preferably filled with the electrolyte solution 67, such as 0.154 Molar NaCl (normal saline).

In some embodiments, the cathode or working electrode 72 extends through a first tube 76 made from any suitable nonconductive insulating material such as polyimide and, for example, having a maximum outer diameter of about, for example, 0.005 inch, a maximum inner diameter of about, for example, 0.004 inch and a length of about, for example, 8 mm. The cathode or working electrode 72 comprises a small portion of bare platinum wire formed by being exposed to the electrolyte solution 67 in main chamber 66. This cathode or working electrode 72 protrudes slightly from an encapsulant 77 of an insulator such as sealing glass or an insulating adhesive. If sealing glass is used as an insulator, a bead of sealing glass can be fused near the distal end of the bare portion of the platinum wire so that the wire extends through the glass bead, near the center, protruding beyond the glass bead. The platinum wire diameter can range from, for example, 0.001 inch to 0.004 inch, and, in some embodiments, is 0.002 inch, and protrudes from 0.1 to 0.3 mm beyond the encapsulant or the bead of sealing glass. The non-protruding portion of the platinum wire is contained in tube 76. The protruding portion of working electrode 72 is preferably rounded and smoothed by some means such as laser melting. The purpose of this rounding and smoothing is to ensure that there are no sharp edges or splinters to cause unwanted irregularities in the electric field potential around the tip of working electrode 72.

In some embodiments, the proximal end of the working electrode 72 is attached or otherwise coupled to a third electrical conductor 27c, for example by soldering or welding. Alternatively, and preferably, working electrode 72 and electrical conductor 27c are the same platinum wire, and the working electrode 72 is formed by stripping the insulation from electrical conductor 27c at the distal tip. In the present embodiment, the first tube 76 and the proximal portion of the glass bead are embedded within the adhesive encapsulant 37, which additionally seals the proximal end of the first tube 76 as well as sealing the glass bead 77 to the first tube 76. The bare distal end of the working electrode 72 is situated in and exposed to the electrolyte solution 67 within oxygen main chamber 66.

The reference electrode 71 of the oxygen sensor 42 can be formed from a silver wire coated with silver chloride, for example, by dipping the silver wire into molten silver chloride or by any suitable electrochemical process. In some embodiments, the electrode 71 has a diameter ranging from about, for example, 0.001 inch to 0.003 inch and, in preferable embodiments, approximately 0.002 inch. The sensor 52 further comprises a second tube 81 made from any suitable nonconductive material such as plastic and preferably a polymer. The second tube 81 extends along the first tube, substantially parallel to the first tube 76, and is provided with an internal bore 82. In some embodiments, tube 81 can have an outer diameter of about, for example, 0.004 to 0.006 inch, and in preferable embodiments 0.005 inch, an inner diameter of about, for example, 0.003 inch to 0.005 inch, and in preferable embodiments 0.004 inch, and a length of about, for example, 3 to 8 mm. In some embodiments, the length of the second tube is 5 mm. As can be seen, the inner diameter of the second tube 81 is only slightly larger than the outer diameter of the reference electrode 71. Substantially the entire length of the second tube is secured or embedded in the polymer adhesive or adhesive encapsulant 37. The internal bore 82 of the second tube 81 is free of the adhesive encapsulant 37 except at its proximal end; the distal opening of the second tube 81 communicates with main chamber 66 so that solution 67 fills second tube 81 as well as main chamber 66. The proximal end of reference electrode 71, inserted into the proximal end of the second tube, can be secured to a conductor 27d by any suitable means such as welding or soldering. In some embodiments, electrode 71 and electrical conductor 27d are the same silver wire, and the reference electrode 71 is formed by stripping the insulation from electrical conductor 27d along the distal portion and coating the stripped portion with silver chloride, as described herein. The reference electrode 71 extends distally into the second tube 81, in some embodiments extending along the axial centerline of the second tube 81, and the base of reference electrode 71 can be bonded to second tube 81, at the same time sealing the proximal end of second tube 81.

Counter electrode 73 can be made from any suitable conductor and can be formed from a platinum wire having a diameter ranging from about, for example, 0.001 inch to 0.004 inch and approximately 0.002 inch. In this embodiment, electrode 73 has a first or proximal portion 82a electrically coupled to a conductor 27e by any suitable means such as soldering or welding. Alternatively, electrode 73 and electrical conductor 27e can be the same platinum wire, and the electrode 73 can be formed by stripping the insulation from electrical conductor 27e along its distal portion. The proximal portion 82a extends along the first tube 76, and can be parallel to the tube 76 and on the opposite side of the first tube from second tube 81. Electrode 73 has a second or central portion 82b that forms a curve or loop that extends over to second tube 81, so as to pass near the working electrode 72. This central portion 82b is disposed in oxygen main chamber 66; the center of the loop of electrode 73 is spaced about, for example, 0.1 to 0.5 mm, in some embodiments about 0.25 mm, from the working electrode 72. The electrode 73 is further provided with a third or distal portion 82c that is parallel to the proximal portion 82a, and extends into the distal opening of the second tube 81 and through much of the second tube 81. Proximal portion 82a, central portion 82b and distal portion 82c of electrode 73 are stripped bare of insulation.

In the present embodiment, the tips of reference electrode 71 and counter electrode 73 are contained within second tube 81 and close to each other, but not touching, and in this regard are separated by a distance ranging up to and including about, for example, 1.5 mm and which can be approximately 1 mm. The opposed tips are located a considerable distance from the distal opening of second tube 81, and in this regard the counter electrode 73 extends proximally into the second tube 81 a distance ranging from about, for example, 3 to 7 mm. In some embodiments, the counter electrode 73 extends proximally into the second tube 81 a distance of approximately 5 mm. The tip of counter electrode 73, which is near reference electrode 71, is rounded and smoothed in the same manner as the tip of working electrode 72. Tubing can be any suitable shape, but the present embodiment has straight tubing with openings on both sides. Some embodiments comprise bent pr curved tubing. Some embodiments comprise tubing with at least one plugged end and at least one opening not at an end.

Oxygen gas permeation through the oxygen-permeable membrane, which can be a polymethylpentene membrane, of cannula 13 results in a change in the oxygen concentration in the electrolyte solution 67. Electronic circuitry (not shown), which in some embodiments is disposed within display module 11, maintains the desired potential of 0.70 volts between the working electrode 72 and the reference electrode 71 while measuring the flow of current from the counter electrode 73 to the working electrode 72. The magnitude of this current is proportional to the concentration of oxygen gas in the electrolyte solution 67 within oxygen main chamber 66 which, in turn, is dependent on the partial pressure of oxygen in the blood surrounding the probe 12 at the oxygen sensor 42. The reduction reaction at the working electrode 72 can be described as:

O₂₍g)+2H₂O+4e⁻→4OH⁻

The oxidation reaction at the counter electrode 73 is believed to be the reverse of this. At the reference electrode 71, the oxidation reaction can be described as:

Ag₍ₛ₎+Cl⁻→AgCl₍ₛ₎+e⁻

Some embodiments are adapted to preserve the signal quality of sensors and probes. Migration of positively charged silver ions (Ag⁺) to working electrode 72, which causes signal drift, is inhibited by placing the end of the counter electrode 73 close to, but not in contact with, the opposed end of the reference electrode 71 so as to provide a positive electric field in the vicinity of the reference electrode 71 to repel Ag⁺ ions and by placing the counter electrode 73 and reference electrode 71 in second tube 81, which has a relatively narrow diameter, thus reducing the migration rate for Ag⁺ ions to working electrode 72. In alternative embodiments, such migration is further inhibited by replacing some or all of the electrolyte in the second tube 81 or in the main chamber 66 with the conductive gel, separating the reference electrode 71 from the main volume of electrolyte solution 67 disposed in the oxygen main chamber 66, and thus further reducing the migration rate of Ag⁺ ions to working electrode 72. In general, inhibiting the migration of positively charged silver ions to working electrode 72 minimizes any upward drift in the signal from the working electrode caused by silver deposition on the working electrode.

In some embodiments of oxygen sensor 42, shown in FIG. 5A, a large reference chamber 100 is formed by distal and proximal walls of adhesive encapsulant and the cylindrical walls of cannula 13, such that the inner diameter of large reference chamber 100 is approximately equal to that of cannula 13. The distal adhesive wall of large reference chamber 100 is positioned distal to and very near the proximal end of second tube 81, but in such a way that the adhesive does not enter second tube 81. The proximal adhesive wall of large reference chamber 100 is placed some distance from the distal adhesive wall of large reference chamber 100, at least far enough to accommodate a useful length of the reference electrode 71, which can be, in some embodiments, 1 mm.

In the embodiment shown in FIG. 5, the tip of counter electrode 73 is near the proximal end of second tube 81, preferably emerging slightly from second tube 81. The reference electrode 71 can be placed anywhere in large reference chamber 100, as long as it does not touch counter electrode 73. The purpose of large reference chamber 100 serves to reduce the likelihood of a gas bubble blocking the proximal opening of tube 81 and the path of conductive ions between large reference chamber 100 and main chamber 66.

Although occupying a small axial length of the probe 12 in some embodiments, oxygen sensor 42 maintains a large physical separation between the working electrode 72 and the reference electrode 71, provides a large volume of electrolyte solution, and inhibits the migration of silver ions to the working electrode 72 and thus the buildup of silver precipitate on the working electrode 72. Additionally, only a small and well-defined surface area of the working electrode 72 is exposed to the electrolyte solution 67.

In some embodiments, sensor signal quality is preserved by configuring the sensors and/or probes of one embodiment to function so that they do not continuously consume reactants (such as electrolyte or gas) during their operating lifetime.

As can be seen in the embodiment of FIG. 1, the cylindrical cannula or sleeve 13 of gas permeable material forms a large surface area circumferential window 29 for both the carbon dioxide sensor 41 and the oxygen sensor 42. Such a circumferential window 29 is particularly advantageous as the covering for the blood gas sensor chambers 51 and 66 since it maximizes the permeable membrane area for a given sensor length. In addition to maximizing the permeable membrane area, the circumferential window 29 eliminates the “wall effect” artifact wherein the gas permeable membrane on the tip or one side of a blood gas sensor probe is fully or partially blocked from exposure to the blood when the probe is inadvertently positioned against a vessel wall. Since the functionality of the sensors is primarily affected by the ability of the target analyte in the blood to reach equilibrium with the solution in the chamber, even if the probe is inadvertently placed against a vessel wall, the circumferential window will assure that a gas permeation path into the sensor chambers still exists so that equilibrium is achieved. Therefore, the sensitivity of the oxygen sensor 42 and carbon dioxide sensor 41 in particular to the wall effect artifact is minimized by having a circumferential window comprised of a membrane material that is as highly permeable to the analyte gases, molecules, and/or ions as possible.

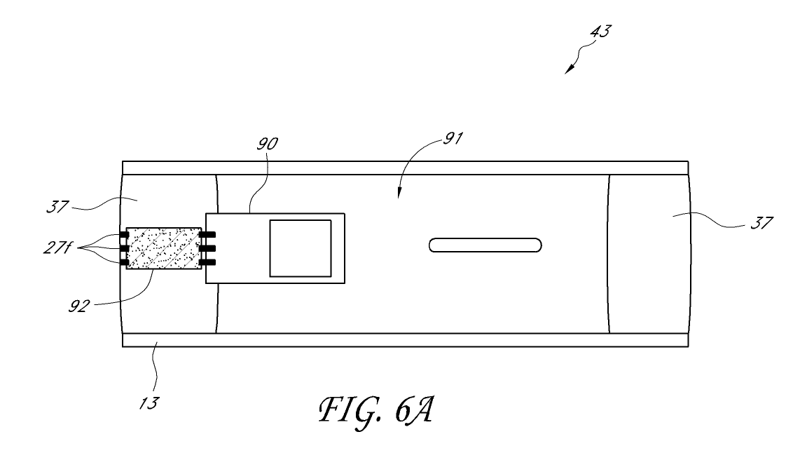

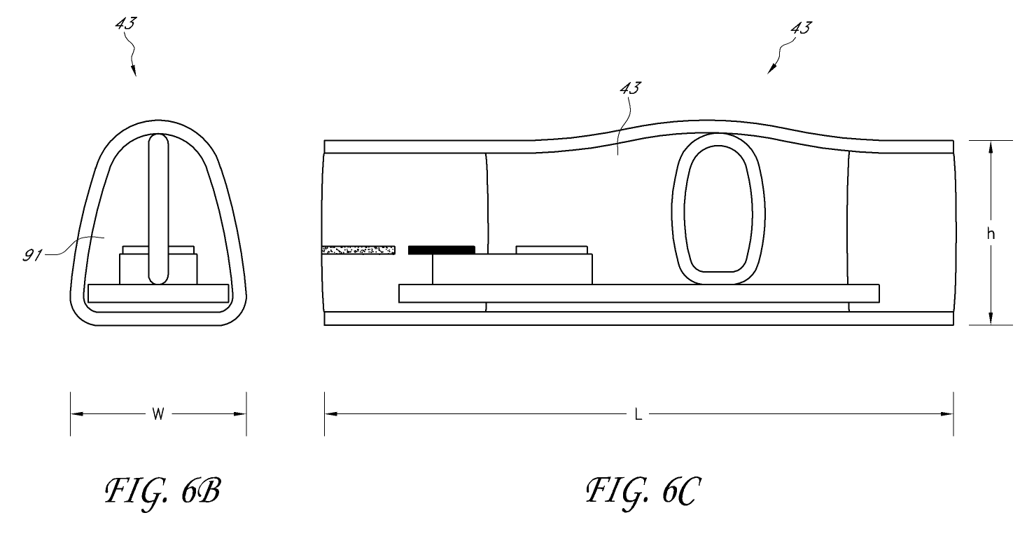

The distal extremity 14b of cannula 13 is further provided with a pressure sensor 43, shown in FIG. 6A. This sensor 43 can be placed either proximal to, or distal to the oxygen or carbon dioxide gas sensor chambers or at any other suitable portion of the probe 12. The pressure sensor chamber 91 is sealed on either end from other chambers with the adhesive encapsulant 33. The connector end of the pressure sensing element 90 is embedded in the proximal encapsulant 33 in order to insulate the connector pads and maintain the placement of the pressure sensor 43 in the chamber 91. The sensing portion of pressure sensing element 90 extends into pressure chamber 91, and is immersed in the fluid filling chamber 91. The diaphragm of the pressure sensing element 90 is fully within the chamber 91, with no part of it touching the adhesive encapsulant 33. This allows it to respond fully to changes in pressure in chamber 91.

The pressure sensing element 90 is appropriately small in size and, for example, can have a length ranging from about 0.020 to 0.100 inch and preferably approximately 0.060 inch, a width ranging from about 0.010 to 0.015 inch and preferably approximately 0.012 inch and a height ranging from about 0.010 to 0.015 inch and preferably approximately 0.012 inch. The length and width and height of the pressure sensing element 90 are shown in FIGS. 6A and 6B.

The pressure sensing element 90 can be of any suitable type, such as of the solid state type manufactured by Silicon Microstructures of Milpitas, Calif. The pressure sensing element 90 is preferably a piezoresistive silicon sensor and, for example, can be a two-resistor, or half-bridge, design using three lead wires. Alternatively, the pressure sensing element 90 is a four-resistor, full-bridge, design using four lead wires. The isolation of the pressure sensing element 90, for example in its own chamber 91, can be advantageous because it cannot function in an ionic solution without a special insulative coating which would dampen its sensitivity. Its chamber 91 is filled with a non-conductive fluid such as silicone oil.

A plurality of conductors 27f extend from the pressure sensing element 90 to respective electrical contacts 18 provided in probe connector 17 to permit electrical communication with the sensor 43 from the proximal extremity of the probe 12. In a preferred embodiment, the conductors 27f are contained within a cover 92. The cover 92 is made from any non-conductive flexible material such as plastic and is optional, but makes some embodiments simpler to assemble.

In order to facilitate transduction of the vessel pressure surrounding the cannula 13 at pressure sensor 43, the effective stiffness of the cannula should be a small fraction of the stiffness of the silicon diaphragm of the pressure sensing element 90. A relatively large area of the cannula relative to the sensor diaphragm and a low modulus of elasticity of the material of the cannula relative to the silicon material of the sensor diaphragm contribute to the effective stiffness of the cannula 13 being a small fraction of the stiffness of the diaphragm of the sensor 43. The stiffness of the wall of the cannula 13 should be low enough that it does not significantly impede the transduction of a pressure change in the bloodstream to the diaphragm of pressure sensing element 90. Thus, the sleeve or cannula 13 in some embodiments provides a substantial portion of the probe strength, particularly in the sensor section 24, where the sensor chambers described herein are filled with liquid.

In addition, in some embodiments, the cross-section of the cannula 13, in the region of the pressure sensor chamber 91, is not perfectly round, but is, for example, oval. A mechanism for causing this shape is shown in FIGS. 6B and 6C and consists of a stretcher consisting of a loop (shown) or a block or plug of some non-conductive material to force cannula 13 to be out-of-round in much of the chamber 91. This helps ensure that the round shape of cannula 13 does not resist a pressure change, but transmits it to a large degree to the fluid filling chamber 91, which in turn transmits the pressure change to the diaphragm of the pressure sensing element 90.

In some embodiments, the pressure sensing element 90 is capable of additionally serving as a temperature sensor, although it is appreciated that any other separate thermocouple, thermistor or other pressure sensor can be provided. If needed, the placement of a separate temperature sensor in close proximity to carbon dioxide sensor 41 and oxygen sensor 42 permits the temperature sensor to accurately reflect the temperature of the surrounding blood. One method to monitor blood pressure and/or other physiologic parameters, such as during a medical procedure, comprises coupling one or more pressure sensors to a central-line catheter side port so that the sensor or sensors are in fluid communication with the blood outside the port.

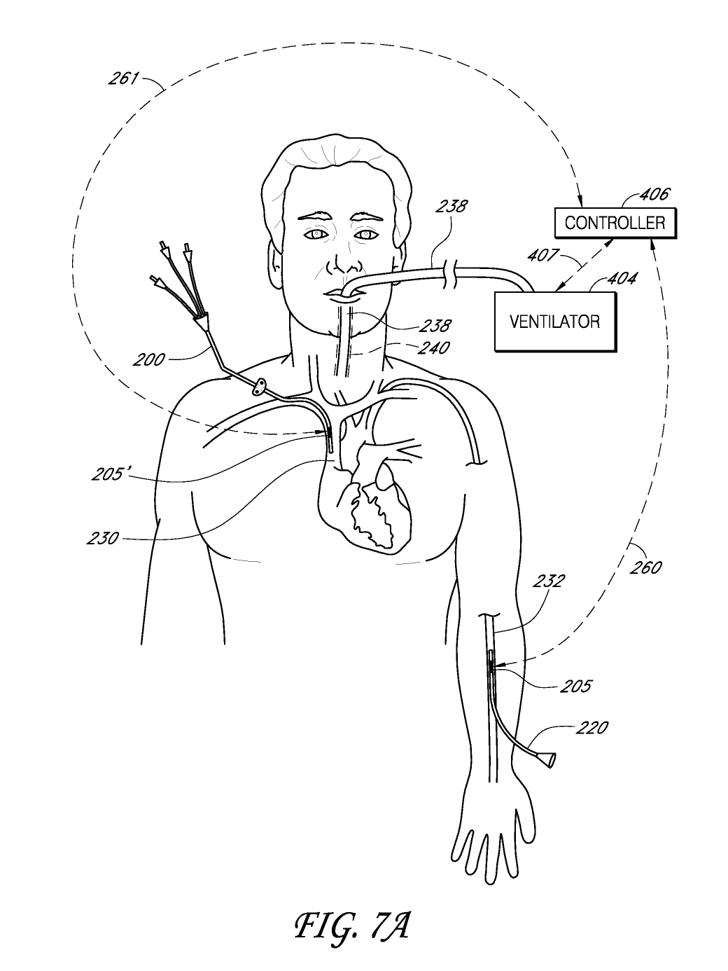

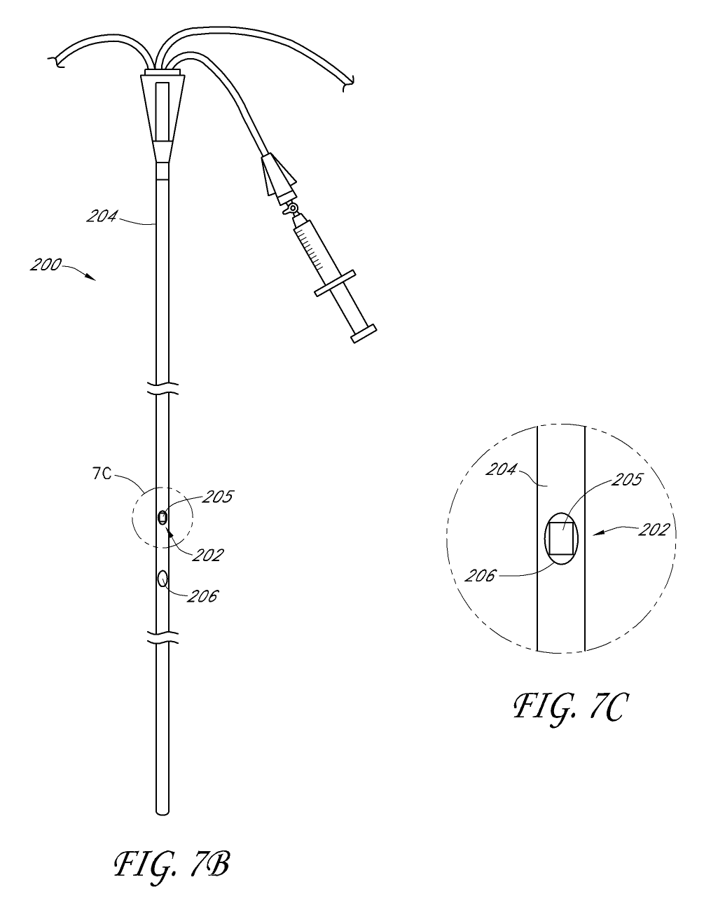

FIG. 7A schematically illustrates various elements of a closed-loop feedback system, according to one embodiment of the invention. Illustrated is a central venous catheter 200 cannulating the right subclavian vein with its distal tip within the superior vena cava 230. A sensor 205′, for example a pressure sensor, is disposed near the distal tip. As described herein, sensor 205′ may be an array of sensors configured to continuously sense pressure, pH, oxygen, carbon dioxide, temperature, and/or other parameters. Also shown is an arterial catheter or guidewire 220 in the left radial artery 232, having a sensor 205 or sensor array attached thereto, also configured to continuously sense pressure, pH, oxygen, carbon dioxide, temperature, and/or other parameters. Sensors 205 and 205′ can communicate either wirelessly or with wires 260 and 261, respectively, to controller 406. In one embodiment, a controller 406 is integral with, operably attached to, or on a module connected with the mechanical ventilator 404, such as via link 407. Alternatively, the controller 406 can be a separate component from the ventilator 404. The controller 406 can control the ventilator 404 at least partially based on inputs from one or both of the sensors 205, 205′. The ventilator 404 can be controlled at least in part as a function of, e.g., one, two, or more of pressure, partial pressure of oxygen, partial pressure of carbon dioxide, pH, and temperature, as well as the oxygen saturation percentage calculated from a hemoglobin measurement of a patient. In embodiments where an oxygen sensor is provided, perfusion can be monitored and used in the control function, based at least partially on either real-time data or data accounted for after a processing or sensing delay, which can be no more than about 0.05, 0.1, 0.2, 0.3, 0.4, 0.5, 1, 2, 3, 4, 5, 10, 15, 30, 45, or 60 seconds, or 1, 2, 3, 4, or 5 minutes in some embodiments. The ventilator 404 can ventilate the patient via an endotracheal tube 238 of which the distal end is placed within the patient's trachea 240.

In some embodiments, arterial blood gas values are transmitted directly to a controller and/or stored in an external memory device. In some embodiments, arterial blood gas values are transmitted wirelessly and stored remotely from the surgical site or stored in a central memory with other data in the treatment environment. In one embodiment, a controller (e.g., processor) is integral with, operably attached to, or on a unit communicating with the mechanical ventilator 404. The controller can control the ventilator at least partially based on inputs from one or both of the sensors 205, 205′, which are blood gas sensors in some embodiments. The controller can, in some embodiments, be configured to provide input back to the sensors, such as to change the frequency of continuous sensing. In some embodiments, such a processor is provided separately from the ventilator 404 (not shown) and may work similarly to control the ventilator 404 based at least partially on sensor 205, 205′ inputs. The ventilator can in turn provide feedback information to the controller in some embodiments. The ventilator 404 can be controlled at least in part as a function of, e.g., one, two, or more of the partial pressure of oxygen, the partial pressure of carbon dioxide, and pH, as well as the oxygen saturation percentage calculated from a hemoglobin measurement of a patient. One embodiment of such a closed-loop feedback system is illustrated in FIG. 7A and described further herein. In embodiments where an oxygen sensor is provided, perfusion can be monitored and used in the control function, based at least partially on either real-time data or data accounted for after a processing or sensing delay.

FIG. 7B shows an example embodiment of a central-line catheter 200 having a side port 202 exiting from an outer cannula 204. A pressure sensor 205 is placed in the side port 202 as shown in FIG. 7C such that it resides distal to the main infusion junction at a proximal end of the catheter (not shown) and herein a flush port 206 that could interfere with the pressure waveform. Once the catheter 200 is inserted and the pressure transducer is immersed in the vessel, waveforms from the blood act on the sensor 205 from the vessel through or at the port access hole 202 and a pressure reading or indication thereof is output from the sensor 205.

In some embodiments of a probe, as shown, for example, in FIGS. 7-12, the various internal wires, conductors and sensors disclosed herein with respect to probe 12 can be wholly or partially replaced with a flexible printed circuit assembly 106 formed from a plurality of layers of a nonconductive substrate. The flexible printed circuit assembly 106 has a length, such as 25 centimeters, appropriate for the assembly to be situated longitudinally within the lumen of a sleeve, such as cannula 13, and has a width ranging from about, for example, about 0.008 inches to about 0.017 inches and preferably about 0.015 inches. More specifically, assembly 106 is formed from first, second, and third layers 107, 121, and 108 of a suitable insulating material such as polyimide. The first layer or flexible substrate 107 has proximal and distal extremities 111 and 112, respectively, and a first or outer planar surface 113 and a second or inner planar surface 114. The third layer or flexible substrate 108 has proximal and distal extremities 116 and 117, respectively, and a first or outer planar surface 118 and a second or inner planar surface 119. The second layer 121 engages the inner surfaces 114 and 119 of the layers 107 and 108, respectively, while providing electrical and mechanical isolation of inner surfaces 114 and 119 from each other.

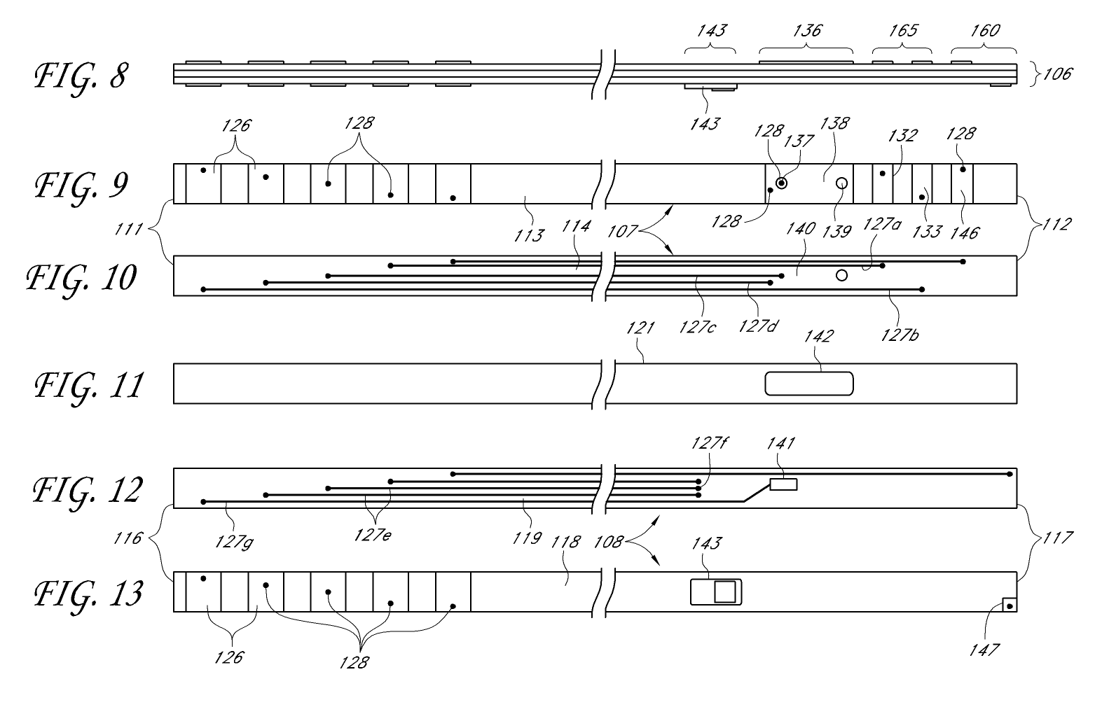

A plurality of contact pads 126 are formed on the proximal extremities of the first and third layers 107 and 108 for forming a low-profile or zero-profile connector, for example similar to connector 17 of probe 12. In this regard, and as shown in FIG. 8, a plurality of five contact pads 126 are formed on outer surface 113 of the first layer 107. As shown in FIG. 12, a plurality of five contact pads 126 are formed on the outer surface 118 of the third layer 108. A plurality of electrodes are formed on the distal portion of the flex circuit assembly 106 and a plurality of conductive traces or conductors 127 are formed on the layers 107 and 108 for electrically coupling the contact pads 126 to respective electrodes. More specifically, and as shown in FIG. 9, a plurality of five conductors 127 extend longitudinally from the proximal extremity 111 to the distal extremity 112 along the inner surface 114 of first layer 107. A plurality of five conductors 127, as shown in FIG. 11, extend longitudinally from the proximal extremity 116 to the distal extremity 117 along the inner surface 119 of third layer 108. As such, the conductors 127 are sandwiched or disposed between the first and third layers 107 and 108 and the insulating second layer 121. The conductors 127 on first and third layers 107 and 108 are electrically connected to respective contact pads 126 by feedthrough vias 128 extending between the outer and inner surface of each of the layers 107 and 108.

The plurality of sensors carried by the distal extremity of the flex circuit assembly 106 includes one or more of a pH sensor 162, a carbon dioxide sensor 160, an oxygen sensor 136, and a pressure sensor 143. Of course, flex circuits can be designed to accommodate any number of circuits that will fit within a given probe or sensor assembly.

A pH sensor assembly, as described in FIG. 3, is attached to contact pads 146 and 147. Contact pad 146 is provided on outer surface 113 of first layer 107 and electrically connected to conductor 127e by means of a via 128. Contact pad 147 is provided on outer surface 118 of third layer 108 and electrically coupled to a conductor 127g on inner surface 117 by means of a via 128.

A carbon dioxide sensor 162, as described with respect to FIG. 4, is attached to contact pads 132 and 133, which are formed on outer surface 113 of first layer 107. The contact pad 132 is electrically coupled to the conductor 127a on the inner surface 114 by means of via 128 and the contact pad 133 is electrically coupled to the conductor 127b on the inner surface 114 by means of a via 128.

An oxygen sensor 136 is additionally provided, as part of the flex circuit layout, and includes a working electrode pad 137 formed on the outer surface 113 of first layer 107 (FIG. 8) and electrically coupled to conductor 127d (FIG. 9) by means of via 128. The sensor 136 includes a counter electrode pad 138 formed on outer surface 113 and electrically coupled to conductor 127c by means of via 128. Thus the working electrode pad is encircled by, but not connected directly to, the counter electrode pad 138. The counter electrode pad 138 is electrically coupled by via 139, extending between the surfaces 113 and 114, to an electrode pad 140 on surface 114. Thus, the counter electrode in oxygen sensor 136 consists of electrode pads 138 and 140 and via 139. A reference electrode pad or reference electrode 141 is included in oxygen sensor 136 and is formed on the inner surface 119 of third layer 108. The reference electrode pad 141 is electrically coupled to conductor 127g. Second layer 121 has a cutout 142 that provides the boundaries of a shallow chamber; the top of this chamber is covered in part by counter electrode pad 140 and the bottom of this chamber is covered in part by reference electrode pad 141. Via 139 is large enough, preferably 0.003 inch in diameter, so that when the three layers 107, 108, and 121 are assembled and the assembly is inserted into a cannula or sleeve as discussed herein and electrolyte solution such as 67 is introduced into the cannula or sleeve, the electrolyte solution such as 67 can easily fill this chamber as well as the volume surrounding oxygen sensor 136.

Flex circuit assembly 106 further includes a pressure sensor 143, preferably including a solid state pressure sensing element like pressure sensor 43 herein, mounted on outer surface 118 of third layer 108 and electrically coupled to three conductors 127f on inner surface 119 by means of three vias 128. As discussed herein, pressure sensor 143 preferably includes a temperature sensor.

The flexible circuit assembly 106 can be mass-produced in a batch process at low cost, thereby minimizing the cost of the multi-sensor probe. In such a batch process, successive layers of conducting materials on insulating substrates, that is layers 107 and 108, are deposited by electroplating, vapor deposition or other methods, then they are patterned by photolithography, laser ablation or other methods. The pads forming contact pads 126 and the various sensors and the traces or conductors 127 of the flexible circuit assembly 106 are primarily formed of copper. The pads are plated with various metals including silver, platinum and gold to create the electrodes of the various sensors or contact pads for attaching the carbon dioxide sensor, the pH sensor and the blood pressure sensor. The contact pads 126 are plated with gold to provide reliable electrical contact with the mating connector of the display module 11. The contact pads 132 and 133 are plated with gold to provide reliable surfaces for attaching a carbon dioxide sensor 41. The working electrode 137 for the oxygen sensor 136 is preferably formed by masking a platinum-plated pad electrode with an insulating material to define a small exposed area of platinum metal in the range from 0.001 to 0.008 inch in diameter and preferably approximately 0.002 inch in diameter. The reference electrode 141 for the oxygen sensor is electrochemically plated with silver chloride.

The contact pads 146 and 147 are plated with gold to provide reliable surfaces for attaching a pH sensor. In addition to or as an alternative to the temperature sensor in pressure sensor 143, the flexible circuit assembly 106 can support a temperature sensor in the form of a patterned thin film of known material forming a temperature-sensitive resistor on the inner surface of one of the layers 107 and 108, or the temperature sensor can be a diode, thermistor, or thermocouple bonded to one of the flexible circuit layers 107 and 108. The patterned layers 107, 121 and 108 are bonded together with insulating adhesive to complete the multi-layer flexible circuit assembly 106.

Once the processing steps have been completed from sheets of substrate materials that have been patterned and adhered in the manner discussed herein, individual circuit assemblies are cut from the sheets. The individual circuit assemblies are thus formed into narrow strips, for example having a width of 0.015 inch, such that each circuit assembly 106 can be inserted into a cannula or sleeve 151, substantially similar to cannula or sleeve 13, and filled with an adhesive encapsulant 33 and electrolyte solutions or other liquids of the type discussed to form the sensor chambers (described herein) in the sensor section 152 of the flexible circuit assembly 106. FIG. 13 illustrates a flexible circuit assembly 106, including various electrodes such as sensors 131, 136, 143 and 147, inserted into the lumen or bore of the cannula or sleeve 151. The proximal end or portion of the flexible circuit 106 includes buried traces or conductors 127 and gold-plated pads 126 which serve as conductors and contacts for the low profile connector 153 of probe 154, which is much like low profile connector 17 discussed herein. The buried traces conduct electrical signals from the sensor electrodes or sensor pads to the electrical contacts pads 126, which serve as a low profile electrical connector 153 that can be coupled to the mating connector 166 of the display module 11.

As described herein, at least the portion of the cannula 13 or 151 that forms the external surface of the respective probe is preferably provided with a durable surface treatment 49, a portion of which is shown in FIGS. 4 and 5, to inhibit the accumulation of thrombus, protein, or other blood components, which might otherwise impair blood flow in the artery or impede the transport of oxygen or carbon dioxide through the circumferential window 29 into the sensing chambers 51 and 66. One preferred method for treating the surface of the cannula or sleeve 13 or 151 is photoinduced graft polymerization with N-vinylpyrrolidone to form a dense multitude of microscopic polymerized strands of polyvinylpyrrolidone, covalently bonded to the probe outer surface. This surface treatment 49 is durable, due to the strong covalent bonds, which anchor the polymer strands to the underlying substrate. Procedures for surface treatment of the polymer cannula or sleeve material are described in copending application Ser. No. 10/658,926 filed Sep. 9, 2003, which is hereby incorporated by reference in its entirety as if set forth fully herein.

The surface treatment 49 adds only a sub-micron thickness to the probe body 13 or 151, yet it provides a hydrophilic character to the probe surface, rendering it highly lubricious when hydrated by contact with blood or water, thereby facilitating the smooth passage of the probe 12 or 154 through the blood vessel. This hydrophilic surface treatment 49 also inhibits the adsorption of protein onto the surface of the underlying polymer substrate, thereby minimizing the accumulation of thrombus, protein, or other blood components on the probe. Although the dense multitude of polyvinylpyrrolidone polymer strands shields the underlying outer wall of the sleeve or cannula from large protein molecules, it does not significantly impede the migration of small molecules such as oxygen or carbon dioxide through the wall of the cannula. Therefore, the surface treatment 49 of the polymethylpentene cannula or sleeve 13 or 151 facilitates consistent, reliable communication of the gases in the blood, such as oxygen and carbon dioxide, through the circumferential window 29 into the carbon dioxide and oxygen sensor chambers 51 and 66, even after prolonged residence time up to three days in the bloodstream of a patient.

The display module 11, as shown in FIG. 1, includes a housing 161 formed of a suitable material such as plastic and which is sized so that it can be worn on the patient, such as on the patient's wrist, arm or other limb, sometimes referred to herein as the subject, with the probe 12 or 154 inserted into vessel(s) in the hand, wrist, forearm or other peripherally accessible vessel. The module 11 also includes a display 162 such as a liquid crystal display (LCD) for displaying measured parameters and other information, and adapted to be readily visible to the attending medical professional, sometimes referred to herein as the user. The display 162 may include backlighting or other features that enhance the visibility of the display. A band 163 attached to the housing 161 is adapted to secure the display module 162 to the subject's wrist. Alternatively, the module II may be attached to the subject's arm or to a location near the subject. Optionally, in the case the subject is a newborn infant (neonate), the module 11 may be strapped to the subject's torso, with the probe 12 or 154 inserted into umbilical vessel(s). The band 163 is comprised of any suitable material, such as Velcro or elastic. Buttons 164 or keys facilitate entry of data and permit the user to affect the display 162 and other features of the module 11. While FIG. 1 shows three buttons, any number or type of buttons, keypads, switches or finger-operable elements may be used to permit entry of parameters or commands, or to otherwise interface with the apparatus 10. Alternatively, there may be no buttons for affecting the display 162, in which case the various screens 162 would appear automatically, in sequence one after the other, at a rate consistent with medical practice. For example, each screen 162 might appear for 3 seconds before it was replaced by the subsequent screen. The module 11 may also include wireless communications capability to facilitate display of physiologic parameters on a remote monitor or computer system, and/or to facilitate the entry of patient parameters or other information into the module 11 from a remote control panel or computer system. The module 11 also includes one or more connectors 166 that provide physical connection and communication with one or more probes 12 or 154. Preferably, each connector 166 includes a receptacle adapted to receive, secure, and communicate with a corresponding connector 17 or 153 on the proximal end of the respective probe 12 or 154.

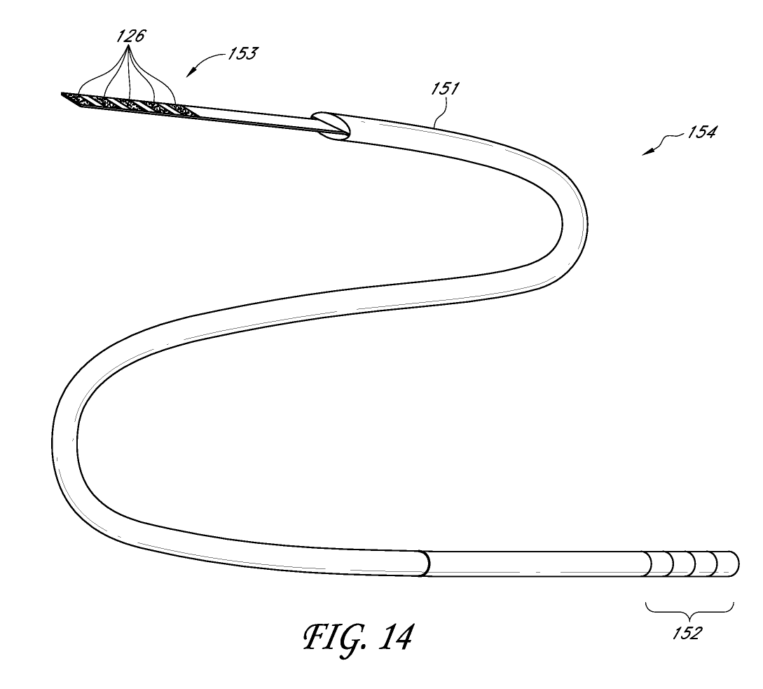

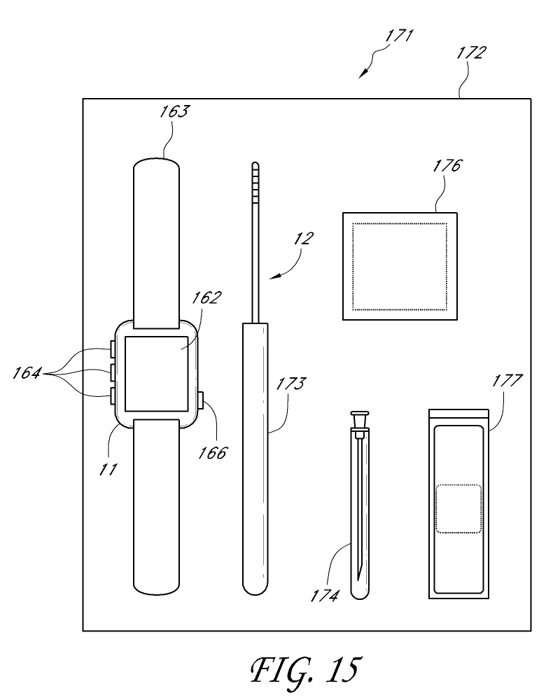

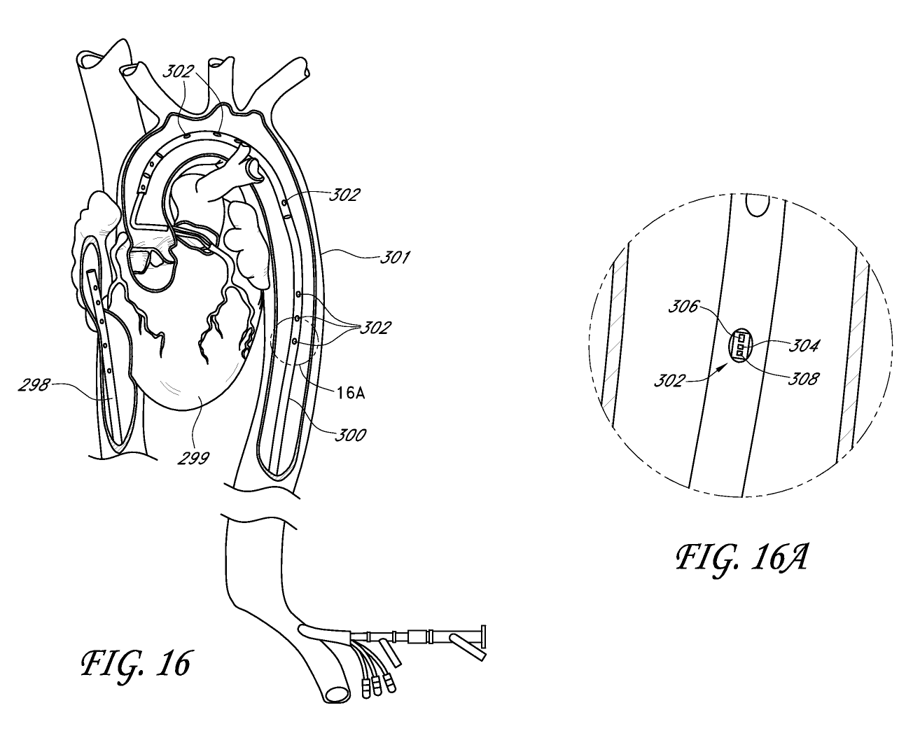

In a preferred embodiment of the display module 11, the module is designed to be low in cost so that it can be packaged together with one or more probes 12 or 154 and accessories as a disposable kit 171, with all of the components of the kit packaged together in a sterile pouch or other container 172, as illustrated in FIG. 14. In addition to the display module 11 and one or more probes 12 or 154, the kit 171 would optionally include a probe holder 173 to protect the probe from damage or degradation, a wrist band 163 or other means for attaching the display module to a patient, a needle or other introducer 174, alcohol swabs 176 for cleaning the skin prior to cannulating the vessel and for cleaning blood or other residue from the probe connector prior to attaching the probe to the module, a bandage 177 to cover the puncture site and anchor the probe in place, and any other items that may be utilized for preparing and using the probe and display module 11. The display module 11 is further designed to require low power so that it can operate for the expected lifetime of the device, such as 72 hours, on battery power without the need for battery replacement or connection to an external power source.

Each of the probes 12 and 154 is preferably suited to be a single-use, disposable device, since it has a limited operational lifetime and is used in direct contact with the subject's blood. The module 11 is durable enough to be used many times, however, the advantage of a disposable module is that it eliminates the expense and the infection hazard associated with cleaning, replacing batteries, and reusing a single module for multiple patients. An additional advantage of a disposable module 11 packaged together with its associated probe is that the calibration data can be stored in the module at the time of manufacture, greatly simplifying the use of the apparatus 10 by eliminating the need for the user to enter calibration data into the module prior to using the probe. A further advantage of a disposable module 11 packaged together with its associated probe is that the calibration data stored in the module at the time of manufacture can account for all of the monitor and probe inaccuracies and artifacts in a single set of calibration coefficients, thereby avoiding the accumulation of inaccuracies that can occur with separate calibrations of the probe and the module 11.

In a first embodiment of the module, no user inputs at all are required, eliminating the need for buttons, keypads, switches and other finger operable elements. In this embodiment, the different display screens shown in FIG. 1 would be shown alternately in an automatically switched sequence designed to best suit the needs of the users. The display module 11 is automatically energized upon connection of the one or more probes 12 or 154 to the module 11, and all of the calibration data and other needed information are pre-programmed into the module at the time of manufacture. Suitable electronic circuitry are included in the display module 11, such as shown and described in co-pending U.S. patent application Ser. No. 10/658,926, filed on Sep. 9, 2003, and incorporated herein by reference in its entirety, for operating the module 11 and the probe coupled thereto. The compact display module 11 makes the most of the wireless communications by freeing the subject from the tubes and cables that normally tether them to their bed, and by eliminating the need for additional bulky instrumentation at the already crowded bedside.

The low-profile connectors 17 or 153 are advantageous in this application since they permit the use of an ordinary hypodermic needle or other suitable introducer 174 to introduce the probe into the blood vessel with minimal trauma to the wall of the blood vessel. The probe 12 or 154 is introduced into the blood vessel by first inserting the appropriately sized hypodermic needle through the skin and into the target vessel. The extremely sharp tip of the hypodermic needle easily penetrates the skin, the underlying tissue, and the vessel wall, while producing minimal trauma. Once the hypodermic introducer needle has entered the target blood vessel, the probe is inserted through the bore of the needle and advanced into the vessel. The blunt tip 26 and the lubricious surface treatment provided on the exterior of cannula 13 or 151 minimize the likelihood of vessel trauma as the probe is advanced within the target vessel. Once the probe is properly positioned within the target vessel, the introducer needle is withdrawn from the artery and the skin, and completely removed from the probe by sliding it off the proximal end of the probe over the low profile connector, leaving the probe in place in the vessel. The low profile connector at the proximal extremity of the probe is connected to connector 166 of the display module 11. During operation, and as shown in FIG. 1 in the first screen of display 162, the arterial blood gas panel that includes oxygen, carbon dioxide, pH, bicarbonate and blood pressure readings can be displayed and thus monitored by apparatus 10. The bicarbonate reading is derived from the circuitry within module 11 from the carbon dioxide and pH readings taken at the sensor section of the probe. Additionally, as shown in the second screen of display 162, shown alongside the module 11, cardiac output, cardiac index, systemic vascular resistance, heart rate and mean arterial pressure readings can be displayed and monitored. Cardiac output is determined from the difference in venous and arterial oxygen concentration. Systemic vascular resistance is determined from cardiac output and blood pressure. The heart rate is the number of heart beats per minute, determined from the data provided by the pressure sensor, and the mean arterial pressure is determined from the systolic and diastolic blood pressure.

In a second embodiment of the display module 11, a minimum number of user input devices are provided so that patient weight, height, hemoglobin and/or hematocrit values can be entered. This will enable the display of cardiac index, as well as a more accurate value of cardiac output.

The small puncture left by the hypodermic needle quickly seals around the body of the probe, thereby preventing excessive bleeding. The puncture site is covered with a bandage 177 and tape to guard against infection and to anchor the probe. Any blood residue on the low profile connector 17 or 153 or the exposed portion of the probe is wiped away with a moist pad or alcohol swab, and the probe connector is then attached to the mating connector 166 on the display module 11. Although the probe has been described for use in a blood vessel, it is appreciated that probes embodying the present embodiment can be introduced into other vessels, lumen or tissue of a body of a patient, by means of any suitable introducer.

The sensors, probes, and methods of the embodiments described herein and in the documents incorporated herein by reference and other sensors, probes and methods, can allow for measurement of blood gases and other characteristics of a subject, such as oxygen and carbon dioxide, as well as other blood parameters including temperature, metabolites, pH, and pressure. A single probe may include one or more sensors, e.g., an oxygen sensor, a carbon dioxide sensor, a temperature sensor, a pH sensor and a pressure sensor. In some embodiments, the sensors are included in a probe body, for example having a small diameter of less than about 0.023 inches so that it can be readily inserted through a 20-gauge needle into a blood vessel, for example, a vessel in the hand, wrist, or forearm. This probe includes at least one sensor with a window 29 having a large surface area and high permeability to the target gas molecules, which facilitates the rapid diffusion of blood gases into or out of the sensor chamber to ensure a fast response to changes in the blood gas concentration. Some embodiments have circumferential, semi-permeable windows, for example, through which a sensor or sensors are in communication with the sensing environment. The circumferential window preserves access to the sensing electrode and provides assurance that the diffusion pathway to the sensor is substantially the same regardless of the sensor's axial orientation with respect to the sensing environment. Similarly, some embodiments offer more stable sensing in environments where rotation or movement of the sensor is inconvenient, difficult or impossible to manage.

The probes utilized are preferably blunt tipped and atraumatic to the vessel wall and are preferably provided with an antithrombogenic surface treatment to inhibit the formation of thrombus or the adhesion of protein or other blood components, ensuring consistent performance of the blood gas sensors and minimizing the need for continuous infusion of heparin to maintain a clot-free environment. The probe carries electrical signals from the sensors, through electrical conductors, to a low profile or other connector removably attached to a mating connector on the display module. The low profile of the preferred connector facilitates the removal of the hypodermic needle or other introducer used to most simply introduce the probe into the lumen of a vein or artery, thereby eliminating the need for using a split sheath introducer or other more complex technique for introducing the probe into the vessel. The display module is small and inexpensive, and it is particularly suited for attachment to the patient's wrist. The apparatus and method herein described may be adapted to the particular requirements of a variety of different medical applications, such as soft tissue and vessel sensing, several of which are outlined herein.

Some embodiments are particularly useful for determining parameters such as pressure, oxygen perfusion, pH (acidity), and/or the concentration of lactate, glucose, potassium, magnesium, etc. in organ tissue such as breast tissue, heart tissue, brain tissue, and/or lung tissue. Some embodiments are adapted to be used during or after revascularization procedures. A benefit of the small size and flexible configuration and arrangement of sensors in the herein-described embodiments is that single or multiple sensors and/or probes can be disposed in or coupled to various available medical implements such that they need not be inserted independently of other instrumentation or peripherally to a treatment site. For example, in certain embodiments, the sensor can be coupled to catheters such as Foley catheters, peripherally-inserted central catheters, and central venous catheters being inserted into a patient for a medical procedure. Analogously to the sensor placement described herein with respect to a pressure sensor 205, placement is carefully chosen to optimize sensor performance and to minimize interference from the ongoing medical procedure such as port flushing or infusion operations. Often, the embodiment can be situated so that the sensor portion communicates with tissue at or near the distal tip of a catheter, such as in the manner shown and described in relation to FIG. 7B. This configuration is especially advantageous when the distal tip or a catheter port is disposed near or at a treatment site, such as a revascularization site during percutaneous coronary revascularization procedures, and there is sufficient space between the catheter wall and the surrounding vessel or tissue wall to allow the target analyte to access the sensor's or probe's sensing electrode through, for example, a semi-permeable membrane.

### Clinical Applications

Some non-limiting examples of clinical applications for the probes and sensors, for example as described herein, will now be described.

### Continuous Arterial Blood Gas Monitoring

For patients, such as those in the intensive care unit (ICU) or coronary care unit (CCU), or intra- or post-operative patients, there is often the need for monitoring arterial blood gases (oxygen and carbon dioxide), pH, and systolic and diastolic blood pressure. Arterial lines are typically small catheters (e.g., 20-gauge) inserted into an artery such as the radial, ulnar, brachial, axillary, subclavian, or femoral artery. Currently, this monitoring is performed on an intermittent basis, such as two, three to twelve, or more times per day, by drawing a blood sample from an arterial line in, for example, one of the aforementioned arteries, placing the sample on ice, and delivering the blood sample to a blood gas analyzer. Untimely delivery of the sample, inadequate sample volume, or clotting can lead to inaccurate results. A multi-sensor probe providing continuous oxygen, carbon dioxide, pH, and pressure measurements, for example as described herein, can eliminate the need and the associated expense and risks of placing and maintaining an arterial line and repeatedly drawing blood samples therefrom. Furthermore, the continuous monitoring provided by continuous devices, for example as described herein, can allow rapid feedback regarding the effects of any interventions such as adjustments to the ventilator settings or administration of drugs. The timely feedback on the effects of the medical interventions permits the subject to be more quickly weaned from the ventilator and transferred out of the critical care unit, a benefit to both the patient and the healthcare system. As a further benefit for the patient, some embodiments of the disclosed indwelling blood gas sensor are particularly useful when connected to a wristband or other display to continuous display blood gases (breath-to-breath).

### Cardiac Output Measurement and Diagnosis and Treatment of Congestive Heart Failure

In a subset of critical care patients, where there is a need to monitor cardiac output, the addition of a venous oxygen sensor probe to the previously described multi-sensor arterial probe, makes it possible for the present embodiment to estimate the cardiac output using a modified arteriovenous oxygen concentration difference equation (the Fick method). Cardiac output, or another value calculated or estimated based upon feedback information from the sensors regarding one or more physiologic parameters could be calculated manually, by a controller configured to adjust the therapeutic settings on a therapeutic device, or a module either distinct or part of the controller itself. Currently, cardiac output is most frequently monitored using the thermodilution technique, which requires placement of a Swan-Ganz catheter in the jugular vein, through the right atrium and right ventricle, and into a branch of the pulmonary artery. The thermodilution technique requires injections of cold saline boluses at intervals, whenever a cardiac output reading is desired. The replacement of the right heart catheter with continuous probes, for example as described herein, greatly reduces the risk to the patient by eliminating the right heart catheterization procedure, and it provides greater utility by providing on-demand cardiac output readings without cumbersome thermodilution measurements.

In another subset of ICU/CCU patients, where there is a need to frequently monitor cardiac output but not necessarily arterial blood gases, a simpler apparatus is a single venous oxygen probe used to monitor the venous oxygen content. This value is combined with independent measurements or estimates of arterial oxygen saturation from a noninvasive pulse oximeter, hemoglobin density from a collected blood sample, and calculated oxygen consumption according to the standard approximation, to calculate cardiac output according to the Fick method. The probe is placed in a vein, such as in the hand, using an experimentally determined compensation factor to account for the expected difference between the oxygen saturation in the right atrium and the oxygen saturation in a vein of the hand. Alternatively, the oxygen probe can be inserted directly through the jugular vein in the neck, into the vena cava or the right atrium of the heart to provide a direct measurement of the oxygen saturation of the mixed venous blood without the need for a compensation factor.

In Fick's original method, the following variables can be measured:
VO₂ (oxygen consumption, mL of gaseous oxygen per minute—may be measured by a spirometer and a carbon dioxide absorber)
Cᵥ (oxygen content of blood from the pulmonary artery—deoxygenated mixed venous blood)
Cₐ (oxygen content of blood from a peripheral artery—oxygenated arterial blood)
   Cardiac output can thus be calculated as equal to VO₂/(Cₐ−Cᵥ).

If no direct arterial oxygen content measurement can be performed, the oxygen content of the blood can be estimated by the following formula:

Oxygen content of blood=(hemoglobin in g/dL)×(1.36 mL O₂/gram of hemoglobin)×(saturation of blood)/100+0.032×partial pressure of oxygen in Torr (mm Hg)

Venous oxygen content can also be measured by the same formula if a venous probe or sensor is not available. The above calculation can be performed manually, or via a software and/or hardware module within the system, and an updated result presented on a display. Besides its utility for estimating cardiac output, the venous oxygen content can be a valuable parameter on its own for assessing the status of the patient.

In some embodiments, a system configured to calculate cardiac output utilizing one or more embodiments incorporating a continuous sensor, for example as described herein, can be used for the diagnosis and or assessment of treatment efficacy of congestive heart failure. Congestive heart failure results in inadequate pumping of the heart resulting in decreased blood flow to peripheral organs and tissues. Implantation of a left heart and/or right heart device incorporating a continuous sensor, for example as described herein, to continuously measure variables to calculate cardiac output can be highly advantageous when titrating congestive heart failure medications in a critical care setting to the cardiac output, such as chronotropic, inotropic, or other agents such as dopamine, dobutamine, isoproterenol, epinephrine, aminone, milrinone, digoxin, beta-blockers, ACE inhibitors, ARBs, vasodilators such as nitroglycerin or hydralazine, diuretics such as thiazide diuretics, loop diuretics, spironolactone, etc. In some embodiments, the arterial and/or venous sensors could determine the respective blood oxygen content and a processor could calculate the cardiac output. A controller could then initiate, discontinue, or titrate the dose of one or more medications housed in an automated medication infusion system operably connected to a venous access line of a patient continuously or at a specified interval according to the cardiac output calculation in accordance with a predetermined algorithm, as well as other parameters such as blood pressure and heart rate, which can also be measured by continuous sensors, for example as described herein. Such a continuous cardiac output monitoring system could be highly advantageous in cardiac step-down and other inpatient units as well as outpatient units, where use of a conventional Swan-Ganz catheter to measure cardiac output would be impractical as well as contraindicated.

### Neonatal Care Applications

Further embodiments of the continuous sensors, for example as described herein, can be used in neonatal care applications. In neonates, there is frequently the need for arterial and venous blood gas monitoring, along with the measurement of cardiac output and other blood parameters. Many continuous sensor embodiments are particularly suitable for neonates, since it minimizes if not eliminates the need for drawing blood from the neonate subject with a small blood volume to draw from. The addition of hemoglobin, bilirubin, electrolyte, or glucose sensors to the blood gas and pH and pressure sensors, for example as described herein, can increase the utility of the multi-sensor probe for this application. The probes are conveniently inserted into any appropriate vessel, such as umbilical arteries and veins, and the display module is appropriate in size to be strapped around the abdomen or other accessible portion of a neonate. In other embodiments, prior to delivery it may be advantageous to place a probe or sensor for continuous monitoring, for example as described herein, into one or more maternal or fetal vessels, such as a placental artery or vein.

### Congenital or Acquired Cardiac Defects

In diagnosing heart defects in neonate and pediatric patients, as well as in adult patients, there is often a need to sample the oxygen saturation in a variety of locations throughout the chambers of the heart and in the great vessels, such as, for example, one, two or more of the left atrium, left ventricle, pulmonary artery, right atrium, right ventricle, or ascending aorta, aortic arch, or descending aorta. This oxygen saturation data is normally collected in conjunction with an angiographic study of the heart, and it permits the operation of a malformed heart to be more accurately diagnosed, thereby resulting in more appropriate treatment for the patient. Currently, oxygen saturation data is collected by drawing multiple blood samples through a small catheter from a variety of locations throughout the heart and the great vessels. These blood samples are sequentially transferred to a blood gas analyzer to obtain an oxygen saturation reading for each sample. Using continuous sensing technology, for example as disclosed herein, a small oxygen sensor mounted on a probe or guidewire of suitable size such as less than about 0.023 inches in diameter and about 50 cm to about 150 cm in length can be advanced through a guiding catheter to various locations, such as those described herein in the heart and the great vessels to sample the oxygen saturation in vivo, thereby reducing the risk to the patient by eliminating the need to draw a large number of blood samples from a small subject and by reducing the time for the procedure. In some embodiments, conditions such as, for example, atrial septal defect, ventricular septal defect, atrioventricular septal defect, patent ductus arteriosus, Tetralogy of Fallot, or patent foramen ovale could be diagnosed, evaluated, and/or managed at least in part by using continuous probes and sensors, for example as described herein. In one embodiment, a first sensor mounted, for example, as described herein can be delivered into the right side of the heart, such as the right atrium and/or ventricle. A second sensor could be delivered into the left side of the heart, such as the left atrium and/or ventricle. The partial pressures of oxygen measured by the two sensors can be measured, and the oxygen content calculated. The maximal normal difference in O₂ content is 0.5 mL/dL between the pulmonary artery and right ventricle, 0.9 mL/dL between the right ventricle and right atrium, and 1.9 mL/dL between the right atrium and superior vena cava. If the blood O₂ content in a chamber exceeds that of the more proximal chamber by more than these values, a left-to-right shunt at that level is probable. Right-to-left shunts are strongly suspected when left atrial, left ventricular, or arterial O₂ saturation is low (e.g., less than 92%) and does not improve with pure O₂ (fractional inspirational O₂=1.0). Left heart or arterial desaturation plus increased O₂ content in blood samples drawn beyond the shunt site on the right side of circulation suggests a bidirectional shunt. The presence of, for example, a right-to-left shunt causing hypoxemia as evidenced by the sensors noted herein could prompt a procedure to repair the underlying cardiac defect.

Some embodiments of continuous sensors and catheters can be used in conjunction with a congenital heart cardiac catheterization wire. Babies with congenital heart defects frequently undergo a cardiac catheterization diagnostic procedure in which a small catheter is threaded from the femoral artery in the groin to the heart. The catheter is positioned in various locations within the chambers of the mal-formed heart to measure pressure and to draw blood samples for oxygen saturation levels. However, the pressure signals measured from such catheters are often dampened due to the small size of the catheter and the blood samples can be so numerous that the baby may need a blood transfusion prior to going for surgical repair of the heart defect. Using embodiments such as those disclosed herein, the catheter can be smaller than the current 3 Fr. catheter catheterization wire. In this manner, a high fidelity pressure reading, which can be significantly more advantageous than simple dampened-out mean pressure values can be taken. In addition, advantageously, no blood draws would be required to achieve such readings.

### Primary Pulmonary Hypertension

Certain embodiments of continuous sensors could also be very useful for the diagnosis and treatment of pulmonary hypertension. A device incorporating such a sensor can be advanced such that the sensor is in the pulmonary artery and configured to continuously monitor, for example, the pulmonary arterial pressure. The dose of a medication, such as a continuous-infusion vasodilator such as a prostacyclin analogue such as treprostinil or epoprostenol, could be titrated in a closed-loop circuit using feedback from the pulmonary arterial pressure sensor. Additional sensors, such as an arterial oxygen sensor placed in an artery for continuous monitoring, can also be used for both diagnostic oxygen content information as well as for possible medication adjustment as discussed herein.

### Cardiopulmonary Bypass

FIG. 16 shows an arterial perfusion catheter 300 positioned in a patient's aorta 301. An arterial perfusion catheter 300 can be used during a cardiopulmonary bypass procedure where the heart 299 is temporarily stopped using a cardioplegic agent. An external heart-lung machine (not shown) is used to maintain perfusion to other body organs and tissues while the patient's heart 299 and lung (not shown) is bypassed. A venous cannula 298 is placed in, for example, the right atrium, superior or inferior vena cava, or femoral vein to withdraw deoxygenated blood from the body. The blood is then reoxygenated externally and the oxygenated blood can be returned to the body via, for example, the arterial perfusion catheter 300. In some embodiments, a probe comprising pH 304, oxygen 306, and carbon dioxide 308 sensors that can be arranged, for example, as described herein is coupled to or near a port 302 in the wall of the catheter 300 to continuously ascertain characteristics of arterial blood as shown, for example, in FIG. 16A. Any or all of the sensors could be placed in various locations along the arterial perfusion catheter 300, such as proximate the proximal end, distal end, or intermediate locations along the elongate catheter body. In some embodiments, the catheter 300 can be positioned such that one or more sensors are positioned in the ascending aorta, aortic arch, and/or descending aorta. In some embodiments, the catheter 300 has expandable elements such as one or more inflatable balloons (not shown) to isolate various locations such as, for example, one or more of the left ventricle, the aortic root, or the brachiocephalic trunk from the oxygenated blood depending on the desired clinical result. In some embodiments, the catheter 300 can be positioned such that one or more sensors is positioned within a coronary artery. Such a probe can advantageously continuously monitor the effectiveness of the external blood oxygenator without requiring frequent blood draws, and further conserve blood and minimize transfusions. In some embodiments, a similar probe with sensors as described can also be placed in venous cannula 298 as well.

Although FIG. 16 shows an embodiment disposed within a catheter 300 disposed arterially, some embodiments are disposed within catheters, trocars, and the like disposed elsewhere in the body. For example, some embodiments are adapted for ascertaining characteristics of breast tissue such testing for necrosis during an exploratory surgery.

### Pacing Lead Sensors

Further embodiments are well-suited for various vascular and tissue sensing applications. For example, for patients who have undergone cardiac surgery, pacing wires are often left in a cardiac chamber such as the right atrium, or in other embodiments the left atrium, left ventricle, and/or right ventricle so that the repaired heart can be paced immediately if required. Such wires are typically eventually pulled out and the insertion holes in which the wires were placed close by themselves with no ill effect (i.e., no bleeding or tamponade). In another embodiment, these wires can be adapted (or replaced) with certain continuous sensors and devices to provide a valuable tool in the management of these patients. For example, oxygen and pressure levels in the left atrium can be measured. Typically, the pressure measurement is particularly useful because it can reflect the left ventricular end diastolic pressure. This measurement reflects intravascular volume status, and thus how well the repaired heart is pumping along the Starling curve. An abnormally high left atrial pressure typically indicates volume overload, whereas, a low pressure is typically indicates volume depletion. Currently, the art provides no way for making these measurements after surgery. In some embodiments, a small probe wire using the techniques and devices described herein would make or require an insertion incision about the same size as the pacing wires, so a surgeon could place an embodiment of a continuous pressure sensor into the left atrium prior to closing the chest, just as he does with the pacing wires, allowing him to monitor pressure in the left atrium after the chest is closed. Intravenous fluids, pressors, or other medications could be titrated either manually or in a closed-loop feedback system as described elsewhere in the application depending on pressure, cardiac output calculations, etc.

Some embodiments of a continuous oxygen sensor, for example as described herein, can be placed on the atrial pacing leads, such as atrial leads, for better management of patients suffering from congestive heart failure along with arrhythmias. Pacing leads could be temporary postoperative leads or leads configured for permanent implantation. In some embodiments, continuous sensors, for example as disclosed herein, can also be placed on leads of automated implantable cardioverter-defibrillators (AICDs). Among the most common reasons that pacemakers malfunction is improper sensing of electrical waves and the partial pressure of oxygen in the heart by sensors on the pacing electrode. Improper sensing can come from incorrect orientation of the pacing lead and sensor instability, for example. In some embodiments, a probe having an oxygen sensor continuously measures the partial pressure of oxygen in the tissue surrounding pacing leads. In some embodiments, a probe having an oxygen sensor continuously measures the partial pressure of oxygen of blood in or near the heart at an advantageous point along the pathway traversed by a pacing lead, such as through the coronary sinus, right atrium, right ventricle, or left ventricle of the heart. Some embodiments serve to replace sensors provided on pacing leads during manufacture. Some embodiments can be attached to or “plugged into” a pre-manufactured pacing lead. Some embodiments are inserted on a separate probe and integrated with the pacing system without necessarily being directly, physically attached to, or located adjacent to or proximate to a pacing lead. For example, a probe of some embodiments is placed in an underperfused region of the body and perfusion data local to that region is used to monitor or direct pacing.

As another example, during diagnostic procedures, it may be clinically desirable to insert a catheter in the coronary sinus (a large vein that returns blood from the myocardium, such as deoxygenated blood from the coronary arteries, to the right atrium). Embodiments of the continuous oxygen sensors, for example as described herein, can be combined with diagnostic coronary sinus catheters and are useful to reflect the amount of oxygen extracted by the myocardium during cardiac pacing challenges such as electrophysiologic studies (i.e., fast heart rates). These electrophysiologic studies may be done on patients who may need pacemakers or defibrillators.

Some embodiments utilize a probe with a central venous catheter. Embodiments of continuous oxygen and pressure sensors, for example as described herein, can be incorporated into a standard central venous catheter (i.e., a multi-lumen catheter routinely used to measure right atrial pressure and to deliver fluids directly into the right atrium). Some embodiments comprise a sensor or multiple sensors coupled to the wall of a catheter. Sensors that continuously measure oxygen in the right atrium, for example, can provide a very good indication of the cardiac output: the more de-oxygenated the blood, the lower the cardiac output.

Some embodiments also comprise a central venous wire. Patients with heart problems often remain in better condition when their fluids are restricted, but it remains important to precisely monitor cardiac output. Some embodiments of continuous oxygen and pressure sensors, for example as described herein, could be inserted on the same size wire as could be used for the arterial blood gas line (e.g., 0.7 mm). This wire, a longer length than used for the arterial line, could be inserted via the internal jugular vein and threaded down into the right atrium. The technique can be the same as that used for thermal dilution catheters, but some embodiments would be smaller, less invasive, safer, and more accurate. For extremely accurate cardiac output, the information can be linked to the arterial blood gases (i.e., Fick or arterial venous differential calculation). In some cases, this may be more accurate than the peripheral venous embodiment in U.S. Pat. No. 6,616,614, which is incorporated herein by reference in its entirety.

### Neurologic Disease Monitoring

Some embodiments can also be adapted for continuous monitoring of tissue or end-organ perfusion and thus viability. For example, neuro-trauma patients can require monitoring of their intracranial pressure and oxygenation. Instead of, or in addition to using a large intracranial pressure catheter inserted via ventriculostomy and connected to an external pressure transducer as is conventionally done, some embodiments of a continuous indwelling pressure sensor, for example according to the present disclosure, would simplify the pressure sensing apparatus. For example, some embodiments of continuous oxygen, temperature, and pressure sensors, for example as described herein, could fit or be adapted to fit into current intracranial drainage or shunt catheters. Other embodiments involve using a sensing probe or one or more sensors not coupled to a probe in conjunction with an internal jugular catheter or other central catheter to provide diagnostic information, feedback, and/or control signals useful in treating patients with neurologic disease (surgery, stroke, hydrocephalus, pseudotumor cerebri, blunt trauma, etc). For example, such patients have swelling of the brain tissue (cerebral edema). Various medications such as steroids or mannitol could be used to reduce the swelling. Some embodiments are adapted to monitor cerebral edema to allow a treatment provider to optimize dosing, or to warn when a surgical procedure is required to reduce the swelling, such as at a threshold pressure that may indicate a higher risk of herniation. In some embodiments in which a patient is on mechanical ventilation, a closed-loop system for treating cerebral edema is provided. An intracranial pressure probe or other continuous sensor, for example as described herein, could sense an increase in intracranial pressure above a threshold level, which would in turn activate a controller to instruct the ventilator to adjust settings, e.g., hyperventilating the patient, which stimulates a physiologic response to increase cerebral blood flow. In some embodiments, a sensor such as a pressure sensor can be operably connected to a spinal needle or catheter to measure the opening, closing, and intermediate pressures during a lumbar puncture.

### Oxygen Consumption and Metabolic Rate Monitoring

Oxygenation can be measured and monitored with some embodiments of the current sensors or probes. One way to tell how well the brain tissue is coping is to measure the amount of oxygen extracted by measuring the venous oxygen content in the blood leaving the brain (either alone, or in conjunction with the arterial oxygen content to the brain). The brain should consume about 20% (i.e., arterial oxygen content to the brain-venous oxygen content leaving the brain). If the oxygen extraction is less, there could be a shunt, significant infarction, or some other problem which would require intervention. Some embodiments of a continuous oxygen sensor, for example, can be placed on a short wire and placed in the jugular bulb to give continuous oxygenation feedback. This feedback is useful if provided, for example, to a processor or equipment that controls steroid or other drug dosing or alerts caretakers of potential adverse or positive events as a function of oxygen levels. In other embodiments, relatively high oxygen content (indicating low oxygen consumption) measured in a particular anatomical location could indicate hibernating or infracted tissue. Relatively low oxygen content (indicated high oxygen consumption) could indicate a hypermetabolic state such as sepsis, hyperthyroidism, or cancer, for example.

Tissue oxygen consumption is independent of oxygen delivery until a critical oxygen delivery level at a point where consumption is constrained by oxygen delivery. Thus, below a certain delivery level, the venous oxygen saturation will fall rapidly. Thus, venous oxygen content as continuously monitored by a venous sensor or probe, for example as described herein, can be advantageously used as a therapeutic endpoint for cardiogenic shock.

### Gas Sensor Applications

Certain continuous sensors, for example some described herein, need not be immersed in liquid (e.g., a blood vessel) and can operate in a gaseous environment. Some embodiments, especially those having an equilibration time of about 30 seconds or less, may be used instead of current methods to measure exhaled gases in an exhalation pathway or endotracheal tube during exhalation by being mounted, embedded, coupled and/or simply placed in the exhalation pathway. One benefit of measuring expired air is that the amount of carbon dioxide being exhaled can be determined and monitored to assess ventilator tube placement. Certain embodiments described herein add the benefit of continuous or semi-continuous sensing capabilities to the “CO₂ dot” method of measuring expired carbon dioxide whereby a treatment provider places an end-tidal CO₂ monitor, such as a color-changing dot in the exhalation pathway and observes a color change. In some embodiments, a pH sensor is also placed in an exhalation pathway such as an endotracheal tube to provide pH measurements in addition to expired gas measurements. Although described as an alternative, the present embodiment can be used in conjunction with an end-tidal CO₂ monitor. Furthermore, in some embodiments, a sensor mounted on, for example, a guidewire can be advanced distally past the trachea into the bronchi, bronchioles, or alveoli to measure, for example, oxygenation, carbon dioxide, or pH in smaller airways.

### Ventilator Management and Closed-Loop Feedback

Some embodiments can be used to measure intravascular partial pressures of oxygen and carbon dioxide as well as pH. Intensive care applications for some embodiments include determination of initial or subsequent ventilator settings, setting any or all ventilator parameters (such as, for example, ventilator mode (e.g., assist-control or intermittent mandatory ventilation), fraction of inspired oxygen, pressure or volume control, backup rate, tidal volume, positive end-expiratory pressure, pressure support, inspiratory time, expiratory time), adjustment of mechanical ventilation over time, either closed loop, semi-closed loop or with manual intervention, use of arterial blood gases for setting alarm parameters, assessment of ventilator modes, weaning, assessment of ventilator synchrony, or determination of the need for mechanical ventilation.

Some embodiments measure partial pressure of oxygen and carbon dioxide during exhalation and can be used in conjunction with intra-arterial measurements of the same to establish arterial-to-alveolar (A-a) gas gradients. This allows perfusion matching, which involves the distribution of blood flow relative to ventilation in the lungs. In some embodiments, indwelling blood gas sensors that are continuously monitoring, for example, oxygen, carbon dioxide, pH, and temperature are linked to a mechanical ventilator (wirelessly or directly) for closed loop regulation of the mechanical ventilation as described in more detail herein. Continuous A-a gradient monitoring can be useful for initial diagnosis or continuing treatment of, for example, pulmonary embolism.

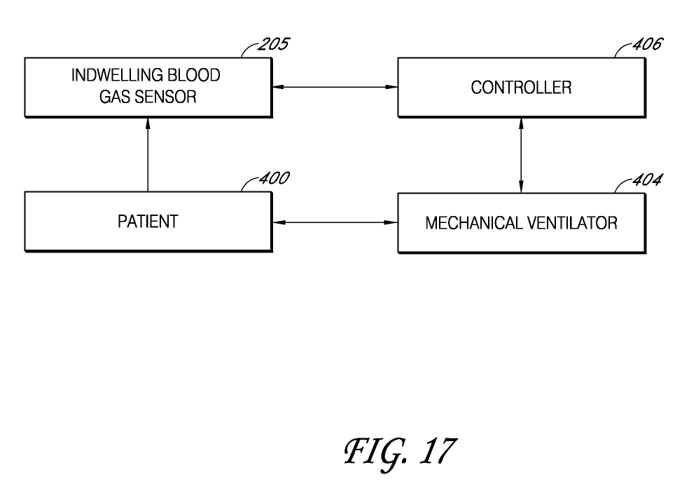

Any and all of the sensors can be linked or any sub-component for closed-loop ventilator regulation. In some embodiments involving a closed-loop system for ventilators, an indwelling blood gas sensor 205, for example, as shown in the schematic of FIG. 17, is inserted into a patient 400. The sensor 205 may be incorporated into the system by way of, for example, a probe 12 of the type shown in FIG. 1 or a catheter-mounted or catheter-embedded sensor 205 as discussed with respect to FIG. 7B. In some embodiments, one or more probes or sensors are coupled to an endotracheal tube as described herein. For example, in some embodiments, the sensors for exhaled CO₂ and pH are placed at the proximal end of an endotracheal tube near the Y-connection in a side-stream sampler space. This placement protects the sensors from the rigors of manual suctioning, etc. In some embodiments, a pH sensor is placed in a ring around the external distal portion of the tube. Sensors or probes can also be coupled to the outer portion of an endotracheal tube and positioned against the tracheal mucosa. Such embodiments are particularly well adapted for monitoring mucosal pH for drops indicating septic conditions and the partial pressure of carbon dioxide in the mucosa, which can indicate perfusion problems characterized by inadequate shunting across the heart to the tracheal mucosa. Measured exhaled CO₂ and measured pH can be compared to arterial blood measurements, such as arterial blood gas measurements. Such a comparison can be done by a computer or an attendant, for example. This information provides for calculating alveolar ventilation.

Referring back to FIG. 17, in some embodiments, one or more sensors 205, which can include, for example, an indwelling blood gas sensor, is configured to measure one or more physiologic parameters of a patient 400 as described herein. The sensor 205 can send real-time and/or delayed information regarding the physiologic parameter to a controller 406, which can then send instructions to a therapeutic device, such as a ventilator 404 to either continue, modify, or discontinue one or more therapeutic settings in response to a predetermined hardware and/or software algorithm as described elsewhere herein. The ventilator 404 would then in turn actually adjust the settings for the patient 400. In other embodiments, the controller 406 could send instructions to a therapeutic device other than a ventilator 404, such as, for example, a device that delivers medication to the patient 400 via any desired route. In some embodiments, the controller 406 could activate a visual, auditory, or other alarm to alert a health care provider if the physiologic parameter falls outside of a specified range. In some embodiments, the ventilator 404 can also send feedback information back to the controller 406. The controller 406 could also send information back to the sensor 205, such as, for example adjusting the sensing frequency.

In some embodiments, arterial blood gas values can be stored in an external memory device. In some embodiments arterial blood gas values are transmitted wirelessly and stored remotely from the surgical site or stored in a central memory with other data in the treatment environment

For example, in one embodiment, upon sensing a pH that is below a certain threshold level (e.g., less than 7.35), indicating an acidosis, the controller 406 could then instruct the ventilator 404 to increase the minute ventilation, e.g., one or both of respiratory rate, tidal volume, or delta-P for pressure controlled ventilation. Upon sensing a pH that is above a certain threshold level (e.g., greater than 7.45), indicating an alkalosis, the controller 406 could then instruct the ventilator 404 to decrease the minute ventilation, e.g., one or both of respiratory rate, tidal volume, or delta-P for pressure controlled ventilation.

In another embodiment, upon sensing a pCO₂ level that is above a certain threshold level (e.g., above 45 mmHg) indicating hypercapnia, the controller 406 could then instruct the ventilator 404 to increase the minute ventilation, e.g., one or both of respiratory rate, tidal volume, or delta-P for pressure controlled ventilation. Upon sensing a pCO₂ level that is below a certain threshold level (e.g., below 35 mmHg), the controller 406 could then instruct the ventilator 404 to decrease the minute ventilation, e.g., one or both of respiratory rate, tidal volume, or delta-P for pressure controlled ventilation.

In another embodiment, upon sensing a pO₂ level that is below a certain threshold level (e.g., below 60 mm Hg) indicating hypoxia, the controller 406 could then instruct the ventilator 404 to increase the FiO₂, minute ventilation, (e.g., one or both of respiratory rate, tidal volume, or delta-P for pressure controlled ventilation), positive end-expiratory pressure, or inspiratory time. Similarly, upon sensing a pO₂ level that is above a certain threshold level, or to reduce the possibility of oxygen toxicity for prolonged ventilation at a high FiO₂, such as at least 50%, 60% or more, the controller 406 could instruct the ventilator 404 to reduce the FiO₂ or adjust other aforementioned parameters. Any of the aforementioned parameters could be adjusted to suit the unique requirements of each patient depending on the desired clinical result. In some embodiments, the feedback loop need not be for invasive ventilation; a controller operably connected to the sensor could adjust non-invasive ventilator parameters, e.g., continuous positive airway pressure (CPAP), bi-level positive airway pressure (BiPAP), intermittent positive pressure breathing (IPPB), and the like to assist in the diagnosis and adjustment of respiratory therapy for pulmonary diseases, e.g., obstructive sleep apnea.

While the preferred embodiment concerns a closed-loop system for controlling a ventilator 404 based on blood gases, such as oxygen and carbon dioxide, other parameters may be accounted for, for example, by adding additional sensors of the types discussed herein and in applications incorporated herein by reference.

The information from the sensor 205 can also be used to determine initial ventilation settings or parameters as described in detail herein. In a closed-loop feedback configuration, the data from the sensor 205 can also be used to adjust mechanical ventilation over time (e.g., manually or automatically via the controller). In still other embodiments or modifications, the data from the sensor 205 can be used for setting maximum or minimum parameters, generating an alarm, assessment of ventilator 404 modes, weaning, assessment ventilator synchrony, or determination of the need for mechanical ventilation.

### Compartment Syndrome

Compartment syndrome occurs when a fixed compartment, defined by myofascial elements or bone, becomes subject to increased pressure, leading to compression of organs and blood vessels. Ischemia, organ dysfunction and eventually organ failure can develop, which is associated with a high mortality rate. Compartment syndrome can occur in any fixed compartment of the body, such as, for example, in the upper or lower extremities, abdomen, or intracranial cavity. While body compartments can be distensible to a certain extent, an endpoint can be reached at which the pressure rises dramatically.

Abdominal compartment syndrome, also known as intra-abdominal hypertension, can be caused by, for example, blunt abdominal trauma or pancreatitis. Intra-abdominal hypertension can be divided into the following 3 categories: (1) Primary or acute abdominal compartment syndrome—this occurs when intra-abdominal pathology is directly and proximally responsible for the compartment syndrome; (2) Secondary abdominal compartment syndrome—this occurs when no visible intra-abdominal injury is present but injuries outside the abdomen cause fluid accumulation; (3) Chronic abdominal compartment syndrome—this occurs in the presence of cirrhosis and ascites, often in the later stages of the disease.

Continuous compartment pressure monitoring of a patient with suspected compartment syndrome can be highly advantageous in rapidly determining when a surgical intervention, such as a decompression procedure, is indicated prior to irreversible organ damage occurring. A continuous probe or sensor carried by a catheter, for example as described herein, can be provided and inserted into the compartment through any desired route, such as percutaneously. The probe or sensor can then be advanced into a cavity, such as the intra-abdominal or retroperitoneal cavity, to measure the intra-cavitary pressure. In some embodiments, it may be desirable to indirectly measure intra-abdominal pressure by measuring the intravesicular pressure of the bladder. If the compartment pressure is greater than a certain threshold level, or if the pressure rapidly rises, the health care provider could be alerted via a display and/or alarm and a decompression procedure considered.

As the herein non-exhaustive examples illustrate, the present disclosure relates to devices, systems, and methods useful in a wide range of applications.

Although certain preferred embodiments and examples have been discussed herein, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described herein, but should be determined only by a fair reading of the present disclosure, including the appended claims.

1. A medical system for continuously measuring a physiologic parameter of a patient and adjusting therapy based upon feedback information on the physiologic parameter, comprising:
a first probe having an elongate body, the first probe configured to be inserted into a first location;
a first sensor operably connected to the first probe and configured to continuously provide feedback information on at least one physiologic parameter at the first location, the physiologic parameter selected from the group consisting of: pH, pCO₂, pO₂, pressure, and temperature; and
a controller operably connected to the first probe,
wherein the controller is configured to receive the feedback information and to adjust a therapeutic setting on a therapeutic device based at least in part on the feedback information.

2. The system of claim 1, wherein the first probe comprises a catheter.

3. The system of claim 1, wherein the first probe comprises a wire.

4. The system of claim 1, wherein the therapeutic device is an infusion device.

5. The system of claim 1, wherein the therapeutic device is a ventilator.

6. The system of claim 1, further comprising:
a second probe having an elongate body, the second probe configured to be inserted into a second location within the patient; and
at least a second sensor operably connected to the second probe and configured to continuously provide real-time feedback information on at least one physiologic parameter at the second location within the patient, the physiologic parameters selected from the group consisting of: pH, pCO₂, pO₂, pressure, and temperature.

7. The system of claim 6, further comprising a module configured to determine the cardiac output of the patient based at least in part from the feedback information from the first sensor and the second sensor.

8. A medical system for continuously measuring a physiologic parameter of a patient and adjusting therapy based upon feedback information on the physiologic parameter, comprising:
a first probe having an elongate body, the first probe configured to be inserted into a first location within the arterial circulation of a patient;
a sensor array operably connected to the first probe and configured to continuously provide feedback information on at least one physiologic parameter at the first location within the patient, the at least one physiologic parameter selected from the group consisting of: pH, pCO₂, pO₂, pressure, and temperature;
a controller configured to operably communicate with the first probe; and
a module configured to calculate the patient's cardiac output based in at least in part upon feedback information from the sensor array,
wherein the controller is configured to receive the feedback information and to adjust a therapeutic setting on a ventilator based at least in part on the feedback information.

9. A method of continuously monitoring at least one physiologic parameter of a patient, comprising:
providing a first probe having an elongate body, the first probe configured to be inserted into a first location, the probe operably connected to a first sensor;
delivering the first probe to the first location;
continuously measuring the at least one physiologic parameter of the patient at the first location using the first sensor, wherein the at least one physiologic parameter is selected from the group consisting of: pH, pCO₂, pO₂, pressure, and temperature; and
transmitting feedback information regarding the physiologic parameter to an output device.

10. The method of claim 9, wherein continuously measuring the at least one physiologic parameter of the patient comprises measuring the physiologic parameter without interruption.

11. The method of claim 9, wherein continuously measuring the at least one physiologic parameter of the patient comprises measuring the physiologic parameter substantially without interruption.

12. The method of claim 9, wherein the first location is within the arterial circulation of the patient.

13. The method of claim 9, further comprising:
interpreting the physiologic parameter feedback information; and
adjusting a therapeutic setting on a therapeutic device at least partially based on the physiologic parameter feedback information from the first location.

14. The method of claim 13, further comprising:
providing a second probe having an elongate body, the second probe configured to be inserted into a second location to measure at least one physiologic parameter of the patient, the second probe operably connected to a second sensor configured to continuously provide feedback information on the at least one physiologic parameter at the second location within the patient;
delivering the second probe to a second anatomical location of the patient; and
continuously measuring the at least one physiologic parameter of the patient at the second location using the second sensor, wherein the at least one physiologic parameter is selected from the group consisting of: pH, pCO₂, pO₂, pressure, and temperature; and
transmitting physiologic parameter feedback information from the second location to a second output device.

15. The method of claim 14, wherein the second anatomical location is within the venous circulation of the patient.

16. The method of claim 14, further comprising determining the patient's cardiac output using the physiologic parameter feedback from the first location and from the second location.

17. The method of claim 13, wherein the therapeutic device is an infusion device.

18. The method of claim 13, wherein the therapeutic device is a ventilator.

19. The method of claim 18, wherein the therapeutic setting comprises FiO₂.

20. The method of claim 18, wherein the therapeutic setting comprises at least one setting affecting minute ventilation.
